(19) United States
(12) Patent Application Publication
(10) Pub. No.: US 2020/0289566 A1
(43) Pub. Date: Sep. 17, 2020

(54) COMPOSITIONS FOR IMPROVING CAR-T CELL FUNCTIONALITY AND USE THEREOF

(71) Applicant: PROSPECT CHARTERCARE RWMC, LLC D/B/A ROGER WILLIAMS MEDICAL CENTER, Providence, RI (US)

(72) Inventor: Sadhak Sengupta, Sharon, MA (US)

(21) Appl. No.: 16/765,776

(22) PCT Filed: Nov. 20, 2018
(86) PCT No.: PCT/US2018/062132

§ 371 Date: May 20, 2020

Related U.S. Application Data
(60) Provisional application No. 62/588,519, filed on Nov. 20, 2017.

(51) Int. Cl. A61K 35/17; C07K 14/54; C07K 14/47; C07K 14/715; A61P 35/00

(52) U.S. Cl. CPC A61K 35/17; C07K 14/5437; A61P 35/00; C07K 14/7155; C07K 14/4748

(57) ABSTRACT

The disclosure relates to compositions and kits comprising CAR-T cells and GSK3β inhibitors, including, use of such compositions and/or kits in the therapy of diseases such as cancer.

### CROSS-REFERENCE TO RELATED APPLICATIONS

The present applications claims the benefit of U.S. Provisional Application No. 62/588,519, filed Nov. 20, 2017, which is hereby incorporated by reference in its entirety for all purposes.

### TECHNICAL FIELD

The disclosure relates to compositions and methods for improving functionality of genetically modified or chimeric antigen receptor T cells (e.g., CAR-T) expressing a receptor protein, which are useful in a variety of therapeutic applications, such as, treatment of tumors.

### BACKGROUND

Use of chimeric antigen receptor expressing engineered T cells (CAR-T), as an immunotherapeutic strategy against malignancies has become a hallmark for successful treatment of peripheral liquid tumors. However, CAR-T therapy in treatment of solid tumors has shown mixed response. Success of adoptive T cell therapy depends upon the ease of access of therapeutic T cells to the antigen source along with co-stimulatory signals, which leads to robust activation profile and strong cytotoxic effects, for example, in hematologic tumors, where CAR-T cells are exposed to copious amounts of malignant B cells in the lymph nodes; or during treatment of highly immunogenic tumors like melanoma. In contrast, during CAR-T therapy of solid tumors, weakly activated T cell resulting from restricted exposure to tumor antigen leads to unstable immune response, anemic clonal expansion and premature clonal contraction.

Various methods to overcome this problem of clonal contraction and weak T cell activation have been employed in the art with moderate success. For example, the CD28 signaling molecule was appended to the intracellular portion of CAR construct to design what are known as second generation CAR-T cells in order to overcome clonal contraction, promote rapid proliferation and to overcome cytokine deficiency. Over time it was observed that this modification did not overcome all barriers to use of CAR-T for solid tumors. Further modifications have been made to create “3rd generation” CARs with added costimulatory molecules like 41BB and/or OX40. In addition, patients are routinely treated with IL2 therapy in order to keep the transferred T cells alive and functioning, resulting in uncontrolled production of cytokines by the therapeutic T cells.

Although these methods have been able to enhance T cell activation to some extent in the treatment of solid tumors, there is still a need for additional innovation to overcome clonal contraction for completely and promote the rapid proliferation and activation of T cells. Such methods are provided herein.

### SUMMARY

The present disclosure is directed to compositions and methods for improving CAR-T therapy. Recognizing that a major impediment in the success of CAR-T cell immunotherapy in solid tumors is weak antigen exposure resulting in less than optimal CAR-T cell activation, which concomitantly leads to weak anti-tumor immune response, the disclosure provides compositions and methods for overcoming the existing hurdles in CAR-T therapy. In particular, the compositions and methods described herein overcome many of the limitations with CD28 and other costimulatory signaling moieties in second-generation CARs, along with cytotoxicity associated with supplementary IL2 therapy.

In various embodiments, a method is provided for ex vivo expansion of a population of T-cells, comprising contacting a population of T-cells with a GSK3β inhibitor. In various embodiments, the T-cells are first transduced with a nucleic acid encoding a chimeric antigen T-cell receptor. In various, embodiments, the T-cells are derived from a mammal. In various embodiments the mammal is a human.

In various embodiments the method further comprises contacting the transduced cells with a tumor antigen.

In various embodiments, a method is provided for ex vivo expansion of a population of T-cells, comprising: isolating a sample comprising said T-cells from a subject; transducing the population of T-cells with a nucleic acid encoding a chimeric antigen receptor protein comprising a molecule that binds to a tumor antigen; and contacting the transduced T-cells with a GSK3β inhibitor.

In various embodiments the method further comprises contacting the transduced T-cells with a tumor antigen. In various embodiments, the T-cells are contacted with a GSK3β inhibitor and the tumor antigen simultaneously.

In various embodiments, the T-cells are transduced with a nucleic acid encoding a chimeric antigen receptor protein comprising interleukin 13 (IL13 CAR-T) or a variant thereof or a fragment thereof. In various embodiments, the nucleic acid encodes the interleukin 13 variant IL13.E13K.R109K or a fragment thereof.

In various embodiments, the nucleic acid encodes a fragment of interleukin 13 comprising a domain that binds to an Interleukin 13 receptor or an extracellular domain thereof or a fusion protein comprising the Interleukin 13 receptor or the extracellular domain thereof. In various embodiments, the tumor antigen comprises an Interleukin 13 receptor (IL13R) or a variant thereof.

In various embodiments, the tumor antigen comprises an alpha (α) chain of Interleukin 13 receptor (IL13Rα) or a variant thereof.

In various embodiments the GSK3β inhibitor is (a) a chemical selected from SB216763, 1-Azakenpaullone, TWS-119 or 6-bromoindirubin-3′-oxime (BIO); and/or (b) a genetic agent selected from micro RNA (miRNA), small interfering RNA (siRNA), DNA-directed RNA interfering (ddRNAi) oligonucleotide, an antisense oligonucleotide or a combination thereof.

In various embodiments the T-cell is a helper T cell, a cytotoxic T cell, a memory T cell, a regulatory T cell, natural killer T cell, or a γδ T cell.

In various embodiments the expanded T-cells are subsequently administered back into a patient in order to treat a disease. In various embodiments, the disease is a cancer. In various embodiments the cancer is a solid tumor. In various embodiments, the tumor expresses a tumor antigen.

In various embodiments, a composition is provided wherein the chimeric antigen receptor protein (CAR-T cell) and a GSK3β inhibitor. In various embodiments the chimeric antigen receptor binds to a tumor antigen.

In various embodiments the T-cell expresses a chimeric antigen receptor comprising interleukin 13 (IL13 CAR-T) or a variant thereof or a fragment thereof. In various embodiments the T-cell expresses a chimeric antigen receptor protein comprising the interleukin 13 variant IL13.E13K.R109K.

In various embodiments the GSK3β inhibitor is a small molecule or a genetic agent. In various embodiments, the GSK3β inhibitor is a small molecule which is SB216763, 1-Azakenpaullone, TWS-119 or 6-bromoindirubin-3′-oxime (BIO); or a genetic agent which is siRNA, miRNA, antisense oligonucleotide, ddRNAi, or a dominant-negative inhibitor of GSK3 (GSK3DN). In various embodiments, the GSK3β inhibitor is a genetic agent selected from micro RNA (miRNA), small interfering RNA (siRNA), DNA-directed RNA interfering (ddRNAi) oligonucleotide, an antisense oligonucleotide or a combination thereof, and dominant-negative allele of GSK3 (GSK3DN).

In various embodiments a formulation is provided for separate administration comprising a T cell, which expresses a chimeric antigen receptor protein (CAR-T cell) and a GSK3β inhibitor.

In various embodiments the GSK3β inhibitor is a small molecule which is SB216763, TWS-119, 1-Azakenpaullone or 6-bromoindirubin-3′-oxime (BIO); or a genetic agent which is siRNA, miRNA, antisense oligonucleotide, ddRNAi, or a dominant-negative inhibitor of GSK3 (GSK3DN).

In various embodiments a kit is provided, wherein the kit comprises in one or more packages, a CAR nucleic acid construct which encodes a chimeric antigen receptor protein comprising interleukin 13 (IL13 CAR-T) or a variant thereof or a fragment thereof; a GSK3β inhibitor; and optionally a first regent for transducing T-cells with said CAR nucleic acid construct; and further optionally, a second reagent for activating T-cells.

In various embodiments, the second reagent is IL13Rα2-Fc. In various embodiments, the nucleic acid construct encodes a chimeric antigen receptor protein comprising interleukin 13 variant IL13.E13K.R109K. In various embodiments, the GSK3β inhibitor is a small molecule which is SB216763, 1-Azakenpaullone, TWS-119 or 6-bromoindirubin-3′-oxime (BIO); or a genetic agent which is siRNA, miRNA, antisense oligonucleotide, ddRNAi, or a dominant-negative inhibitor of GSK3 (GSK3DN). In various embodiments, the GSK3β inhibitor is a genetic agent which comprises micro RNA (miRNA), small interfering RNA (siRNA), DNA-directed RNA interfering (ddRNAi) oligonucleotide, an antisense oligonucleotide or a combination thereof, and GSK3DN.

In various embodiments a T-cell is provided that has inhibited GSKβ expression or activity compared to a native or a wild-type T-cell. In various embodiments, the T cell is a helper T cell, a cytotoxic T cell, a memory T cell, a regulatory T cell, natural killer T cell, or a γδ T cell.

In various embodiments, a method for ex vivo expansion of a T-cell is provided comprising, isolating a sample comprising T-cells from a subject; contacting the T-cells with a GSK3β inhibitor; transducing the T-cells with a nucleic acid encoding a chimeric antigen receptor protein comprising a molecule that binds to a tumor antigen; and contacting the transduced T-cells with the tumor antigen to expand transduced T-cells.

In various embodiments, a method is provided for ex vivo expansion of a T-cell, comprising, isolating a sample comprising T-cells from a subject; contacting the T-cells with a GSK3β inhibitor; transducing the T-cells with a nucleic acid encoding a chimeric antigen receptor protein comprising a molecule that binds to a tumor antigen; and contacting the transduced T-cells with the tumor antigen to activate and/or expand transduced T-cells.

In various embodiments, the T-cells are transduced with a nucleic acid encoding a chimeric antigen receptor protein comprising interleukin 13 (IL13 CAR-T) or a variant thereof or a fragment thereof. In various embodiments, the nucleic acid encodes the interleukin 13 variant IL13.E13K.R109K or a fragment thereof. In various embodiments, the nucleic acid encodes a fragment of interleukin 13 comprising a domain that binds to an Interleukin 13 receptor or an extracellular domain thereof or a fusion protein comprising the Interleukin 13 receptor or the extracellular domain thereof. In various embodiments, the tumor antigen comprises an Interleukin 13 receptor (IL13R) or a variant thereof. In various embodiments, the tumor antigen comprises an alpha (α) chain of Interleukin 13 receptor (IL13Rα) or a variant thereof.

In various embodiments, the GSK3β inhibitor is (a) a chemical selected from SB216763, 1-Azakenpaullone, TWS-119 or 6-bromoindirubin-3′-oxime (BIO); and/or (b) a genetic agent selected from micro RNA (miRNA), small interfering RNA (siRNA), DNA-directed RNA interfering (ddRNAi) oligonucleotide, an antisense oligonucleotide or a combination thereof.

In various embodiments, the T-cell is a helper T cell, a cytotoxic T cell, a memory T cell, a regulatory T cell, natural killer T cell, or a γδ T cell.

In various embodiments, a method is provided for treating a disease that is treatable by adoptive transfer of T-cells in a subject in need thereof, comprising administering, into the subject, an effective amount of a composition comprising a plurality of activated and expanded T-cells wherein the activation comprises contacting the CAR-T with an antigen and the expansion comprises contacting the activated CAR-T cells with a GSK3β inhibitor.

In various embodiments, a method is provided for treating a disease that is treatable by adoptive transfer of T-cells in a subject in need thereof, comprising administering, into the subject, an effective amount of a composition comprising a plurality of activated and expanded T-cells wherein the activation comprises contacting the CAR-T with an antigen and the expansion comprises contacting the activated CAR-T cells with a GSK3β inhibitor.

In various embodiments, the GSK3β inhibitor is (a) a chemical selected from SB216763, TWS-119, 1-Azakenpaullone or 6-bromoindirubin-3′-oxime (BIO); and/or (b) a genetic agent selected from micro RNA (miRNA), small interfering RNA (siRNA), DNA-directed RNA interfering (ddRNAi) oligonucleotide, an antisense oligonucleotide or a combination thereof.

In various embodiments the disease is a tumor disease, a pathogenic disease selected from a bacterial disease, a viral disease and a protozoan disease, or an autoimmune disease

In various embodiments, a method is provided for treating a tumor in a subject in need thereof, comprising administering, into the subject, an effective amount of a composition comprising a plurality of activated and expanded T-cells expressing a chimeric antigen receptor protein comprising a molecule that binds to a tumor antigen (CAR-T), wherein the activation comprises contacting the CAR-T with the tumor antigen and the expansion comprises contacting the activated CAR-T cells with a GSK3β inhibitor, wherein the activated CAR-T cell expresses a chimeric antigen receptor protein and wherein the chimeric antigen receptor protein binds to a tumor antigen.

In various embodiments, the T-cells are autologous T-cells.

In various embodiments the T-cell expresses a chimeric antigen receptor comprising interleukin 13 (IL13 CAR-T) or a variant thereof or a fragment thereof. In various embodiments the T-cell expresses a chimeric antigen receptor protein comprising the interleukin 13 variant IL13.E13K.R109K.

In various embodiments, the GSK3β inhibitor is (a) a chemical selected from SB216763, TWS-119, 1-Azakenpaullone or 6-bromoindirubin-3′-oxime (BIO); and/or (b) a genetic agent selected from micro RNA (miRNA), small interfering RNA (siRNA), DNA-directed RNA interfering (ddRNAi) oligonucleotide, an antisense oligonucleotide or a combination thereof.

In various embodiments, the T-cells are activated and expanded simultaneously or sequentially. In various embodiments the tumor is IL13R positive. In various embodiments, the tumor is an IL13R positive glioma.

In various embodiments, a method is provided for generating tumor-specific memory T cells, comprising transducing T-cells isolated from a subject's biological sample with a nucleic acid encoding chimeric antigen receptor (CAR-T) comprising a molecule that binds to a tumor antigen; contacting the CAR-T cells with the tumor antigen and a GSK3β inhibitor; detecting a first marker specific to memory cells and a second marker specific for the tumor antigen, thereby generating tumor-specific memory T cells.

In various embodiments, the CAR-T cells are transduced with a nucleic acid encoding IL13 or a fragment thereof or a variant thereof. In various embodiments, the CAR-T cells are transduced with a nucleic acid encoding the IL13 variant IL13.E13K.R109K. In various embodiments, the tumor antigen is IL13 receptor or a ligand-binding domain thereof.

In various embodiments, the GSK3β inhibitor is (a) a chemical selected from SB216763, TWS-119, 1-Azakenpaullone or 6-bromoindirubin-3′-oxime (BIO); and/or (b) a genetic agent selected from micro RNA (miRNA), small interfering RNA (siRNA), DNA-directed RNA interfering (ddRNAi) oligonucleotide, an antisense oligonucleotide or a combination thereof.

In various embodiments the T-cells are activated and expanded simultaneously or sequentially. In various embodiments the marker specific for memory cells is selected from CD45RO+ and CD45RA+ and the marker specific for tumor antigen comprises expression of a protein which binds to the tumor antigen. In various embodiments, the CAR-T cells are specific for IL13R-positive tumor cells, as ascertained by a functional assay comprising binding to, and optionally destruction of, IL13R-positive cells. In various embodiments, the memory T-cells are CD8+ T-cells.

In various embodiments, the method further comprises detecting a third marker for memory CAR-T cell homeostasis. In various embodiments, the third marker is expression, T-bet expression, and/or PD-1 expression. In various embodiments, wherein increased T-bet expression and/or attenuated PD-1 expression indicates improved CAR-T cell homeostasis. In various embodiments, T-cell homeostasis comprises reduced T cell exhaustion, sustained cytokine expression, T-cell clonal maintenance, and/or promotion of CAR-T memory development. In various embodiments, the the CAR-T cells generated via activation with the tumor antigen and expansion in the presence of the GSK3β inhibitor demonstrate increased specificity and memory towards tumor cells expressing the tumor antigen.

### BRIEF DESCRIPTION OF THE DRAWINGS

The details of one or more embodiments of the disclosure are set forth in the accompanying drawings/tables and the description below. Other features, objects, and advantages of the disclosure will be apparent from the drawings/tables and detailed description, and from the claims.

FIG. 1 shows that GSK3β inhibition protects activated CAR-T cells from ATCD in the absence of IL2 supplement in vitro. FIG. 1A shows that in absence of SB21763, steady decline in survival of IL13Rα2-Fc -activated IL13CAR-T (open square, solid line; Top panel; p=0.2), which is rescued up to the survival levels of IL2-supplemented IL13CAR-Ts (closed square, dashed line) upon GSK3β inhibition with SB216763 (Bottom panel; p<0.05). Results are representative of 1 of 2 experiments with n=3 wells per sample per time point. Error bars represent SD. FIG. 1B shows flow cytometric representation of frequencies of IL13CAR-T cells expressing FasL upon activation with IL13Rα2-Fc only (Top panel) and with GSK3β inhibition (Bottom panel). Results are representative n=3 independent experiments. FIG. 1C shows a representative FACS profile of CFSE dilution showing IL13CAR-T cell proliferation without any treatment (Top), treated with SB216763 only (Second), activated with IL13Rα2-Fc only (Third), and activated with IL13Rα2-Fc+SB216763 (Bottom).

FIG. 1-supplement shows results of IL13Rα2 specificity of IL13CAR-T cells of the disclosure. FIG. 1A-supplement shows flow cytometric representation of IL13CAR-T enrichment upon coculture with IL13Rα2⁺ U251MG tumor cells at different effector to target cells (E:T) ratio (left); activation with 1 and 10 μg/ml of IL13Rα2-Fc (middle), and IL13Rα1-Fc (right) Untransduced T cells are represented by open lines, while IL13CAR-Ts are closed lines. FIG. 1B-supplement shows flow cytometric representation of CFSE dilution depicting IL13Rα2-specific proliferation of IL13CAR-T cells in presence of U251MG cells (top) at E:T ratio of 1:0 (Black), 1:1 (Grey) and 1:2 (open); upon activation with 0 (black), 1 (grey) and 10 (open) μg/ml of IL13Rα2-Fc (middle) and IL13Rα1-Fc (bottom).

FIG. 2 shows GSK3β inhibition results in T-bet upregulation and decrease in PD-1 expression in activated CAR-T cells. FIG. 2A shows flow cytometric representation of intranuclear T-bet expression in IL13CAR-T cells (left panel); and frequencies of PD-1+IL13CAR-T cells (right panel) upon activation with IL13Rα2-Fc in absence or presence of SB216763. Results are representative n=3 independent experiments. FIG. 2B shows relative expression (qPCR) of TBX21 (T-bet; Left panel) and PDCD1 (PD-1; Right panel) genes in IL13Rα2-Fc activated IL13CAR-T cells. Data was analyzed using 2^{-ΔΔC}^{_{T}} method after normalizing against GAPDH. Error bars represent SEM from N=3 independent experiments.

FIG. 2-supplement shows transduction efficiency of IL13CAR. T cells were enriched with OKT-3 and IL2 from PBMCs harvested from three blinded donors, and transduced three times with IL13CAR-expressing retroviral supernatant to maximize transduction efficiency (TE). Forty-eight hours after final transduction, TE was measured by observing expression of human IL13 on CD3+ T cells using flow cytometry. All experiments in this study were normalized to the TE of IL13CAR to eliminate donor-dependent variations.

FIG. 3 shows GSK3β inhibition results in increased expression of β-catenin in the nucleus of antigen-specific CAR-T cells. Representative histogram profiles of nuclear β-catenin expression in unstimulated (Top panel); IL13Rα2-Fc activated (Middle panel); and SB216763-treated IL13Rα2-Fc activated IL13CAR-T cells (Bottom panel). Treated or untreated IL13CAR-T cells were stained with Rat anti-human IL13 primary antibody/APC anti-rat IgG1 secondary antibody, and rabbit anti-β-catenin MAb/FITC anti-rabbit IgG secondary antibody. Specific antibody controls were used to eliminate background staining. Results are representative n=2 experiments.

FIG. 3-supplement shows results of experiments on CD8 enrichment of IL13CAR-T cells. FIG. 3A-supplement shows flow cytometric representation of CD8:CD4 ratio in IL13CAR-T cells activated with IL13Rα2-Fc+SB216763. Each panel represents FACS profile from each of 3 donors. Gates were drawn on the basis of respective antibody controls. FIG. 3B-supplement shows relative expression of IFNG (Interferon-gamma) genes in IL13Rα2-Fc activated IL13CAR-T cells. Data was analyzed using 2^{-ΔΔC}^{_{T}} method after normalizing against GAPDH. FIG. 3C-supplement shows interferon gamma levels measured by ELISA from culture supernatants of IL13CAR-T cells that were treated with SB216763 alone or in combination with IL13Rα2-Fc activation. Error bars represent SEM from N=3 independent experiments.

FIG. 4 shows antigen-specific CAR-T cell memory phenotype upon GSK3β inhibition. FIG. 4A shows a representative FACS profile of IL13CAR-T cell frequencies that were activated with IL13Rα2-Fc in presence (Right panel) or absence (Left panel) of SB216763. FIG. 4B shows a line graph representation of IL13CAR-T cell memory phenotype. Error bars represent SEM from N=3 independent experiments.

FIG. 5 shows in vivo tissue distribution of CAR-T and expression of T effector memory phenotype in tumor-bearing mice treated with IL13CAR-T. FIG. 5A (left) shows raphical representation of tissue-specific IL13CAR-T distribution in tumor-draining lymph nodes (top), spleens (middle), and tumor-infiltrating lymphocytes (bottom) from tumor bearing animals. FIG. 5B (right) shows CD45RO⁺CD127⁺ IL13CAR-T distribution in tumor-draining lymph nodes (top), spleens (middle), and tumor-infiltrating lymphocytes (bottom) from tumor bearing animals. Tumors were observed in all surviving xenograft animals that were treated with unactivated IL13CAR-T cells (100% recurrent; white circles*), Tumors were detected in 67% of surviving animals (black circles**) that were treated with IL13Rα2-Fc activated IL13CAR-T cells. No tumors were detected in surviving animals that were treated with SB216763-treated IL13Rα2-Fc activated IL13CAR-T cells (0% recurrent; grey circles).

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Other embodiments, features, objects, and advantages of the present teachings will be apparent from the description and accompanying drawings, and from the claims. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Unless otherwise defined, scientific and technical terms used in connection with the present teachings described herein shall have the meanings that are commonly understood by those of ordinary skill in the art.

The present disclosure is directed to compositions and methods for improving CAR-T therapy. Recognizing that a major impediment in the success of CAR-T cell immunotherapy in solid tumors is weak antigen exposure resulting in less than optimal CAR-T cell activation, which concomitantly leads to weak anti-tumor immune response, the disclosure provides compositions and methods for overcoming the existing hurdles in CAR-T therapy. In particular, the compositions and methods described herein overcome many of the limitations with CD28 and other costimulatory signaling moieties in second-generation CARs, along with cytotoxicity associated with supplementary IL2 therapy.

In various embodiments, the compositions and methods of the disclosure are directed to use of adjuvants for improving the survival and/or effectiveness of CAR-T cells. In particular, the disclosure demonstrates that GSK3β inhibitors may be used to increase proliferation, to rapidly expand and also improve survival of antigen-specific CAR-T cells. As demonstrated in detail in the Examples section of the disclosure, pharmacological inhibition of GSK3β promoted antigen-specific CAR-T cell proliferation and long-term survival of these T cells. GSK3β inhibition protected activated CAR-T cells from T cell exhaustion by mitigating PD-1 expression, and further promoted development of specific effector CAR-T memory phenotype that could be modulated with variability in antigen exposure. Treatment of tumor-bearing animals with GSK3β inhibited antigen-specific CAR-T cells resulted in 100% tumor elimination and increased accumulation of memory CAR-T cells in spleens and draining lymph nodes. Tumor re-challenge experiments in animal models resulted in 100% tumor elimination and progression-free survival when treated with GSK3β-inhibited antigen-experienced CAR-T cells. Together, these results demonstrate that this adjuvant-like effect of GSK3β inhibition on activated CAR-T cells provides an effective method for implementing CAR-T immunotherapy against solid tumors.

The data in the Examples of the present disclosure further demonstrate that GSK3β inhibition plays an important role in the successful manipulation of CAR-T cell function. Surprisingly, it was found that activity was restricted to antigen-specific CAR-T cells or those CAR-Ts that were activated with antigen or ligand. GSK3β inhibition not only played a role in activated CAR-T proliferation, but it also promoted CD8+ CAR-T effector memory (TEM) generation. The results demonstrate that GSK3β-inhibition used a combined effect of increased cell division and increased survival of antigen-specific CAR-Ts; however, there were no proliferative effects of GSK3β inhibition on CAR-T cells that were not activated; neither did GSK3B inhibitors have any effect on untransduced T cells that lacked the IL13CAR expression. These observations established the fact that the proliferative-effect of GSK3β inhibition was specific for activated CAR-Ts.

In various embodiments of the invention, GSK3β inhibition results in increased tumor protection of a longer period of time. In various embodiments of the invention GSK3β inhibition results in an increased immunologic memory and expanded and/or proliferated CAR-T cells.-Additionally, the studies with experimental xenograft animals challenged with GSK3β-inhibited antigen-specific CAR-T showed that CAR-T cells treated with GSK3B inhibitors conferred tumor protection for longer periods, which suggests immunologic memory of expanded and/or proliferated CAR-T cells. The studies point to a hitherto unrecognized method of selectively expanding a sub-population of antigen-specific CAR-T cells.

The disclosure accordingly relates to the following non-limiting embodiments:

In various embodiments, the disclosure relates to a method for manipulating a T-cell comprising, contacting the T-cell with a GSK3β inhibitor. In some embodiments, the GSK3β inhibitor is a small molecule chemical agent, e.g., SB216763 (3-(2,4-Dichlorophenyl)-4-(1-methyl-1H-indol-3y1)-1H-pyrrole-2,5-dione), 1-azakenpaullone, TWS -119 or 6-Bromoindirubin-3′-oxime (BIO), and TWS-119. In some embodiments, the GSK3β inhibitor is a genetic agent, e.g., RNA interference (RNAi) via use of, for example, microRNA (miRNA), small interfering RNA molecule (siRNA), a DNA-directed RNA interference (ddRNAi) oligonucleotide, or an antisense oligonucleotide that is specific to GSK3β, as well as dominant-negative allele of GSK3β (GSK3DN). Preferably, the inhibitor inhibits human GSK3β, e.g., human GSK3β variant 1 (mRNA sequence in GENBANK: NM_002093; protein sequence: NP_002084), human GSK3β variant 2 (mRNA sequence in GENBANK: NM_001146156; protein sequence: NP_001139628) or human GSK3β variant 3 (mRNA sequence in GENBANK: NM_001354596; protein sequence: NP_001341525). Yet in some embodiments, GSK3β inhibition comprises deletion or disruption GSK3β, e.g., via targeted knockout. In some embodiments, the manipulation increases expansion, proliferation, survival of T-cells and/or reduces exhaustion of activated T-cells.

Any type of T-cell may be manipulated by the foregoing method, including, but not limited to, T helper cells, cytotoxic T cells, memory T cells (e.g., central memory T cells, stem-cell-like memory T cells (or stem-like memory T cells) or effector memory T cells (e.g., TEM cells and TEMRA cells)), Regulatory T cells (also known as suppressor T cells), Natural killer T cells, Mucosal associated invariant T cells, γδ T cells, tumor-infiltrating T-cells (TILs), and CAR-T cells. Preferably, the T-cell is a helper T cell, a cytotoxic T cell, a memory T cell, a regulatory T cell, natural killer T cell, or a γδ T cell. Especially, the T-cell is a CAR-T cell. In particularly preferred embodiments, the T-cell is an activated CAR-T cell. As is known in the art, CAR-T cells are generally activated using antigen stimulation and the CAR-T cells obtained from such process are antigen-specific, e.g., specific to a tumor antigen such as interleukin 13 receptor (IL13R) or a variant thereof.

In various embodiments, the T-cells are not memory T cells (e.g., central memory T cells, stem-cell-like memory T cells (or stem-like memory T cells) or effector memory T cells (e.g., TEM cells and TEMRA cells).

The disclosure further relates to T-cells, which have been manipulated by the foregoing method, wherein the expression or activity of GSK3β is inhibited, e.g., via use of a chemical or genetic inhibitor as provided above. Preferably, the T-cell has inhibited expression or activity of GSK3β compared to a wild-type or a normal T-cell. Particularly preferably, the T-cell exhibits diminished GSK3β activity compared to a wild-type or a normal T-cell. Especially, the T-cell exhibits diminished GSK3β activity compared to a wild-type or a normal T-cell due to RNA interference via use of siRNA, miRNA, antisense oligonucleotide, ddRNAi, or a dominant-negative inhibitor of GSK3 (GSK3DN).

In some embodiments, the disclosure relates to use of T-cells that have been manipulated or modified in accordance with the methods of the disclosure. Herein, the manipulated T-cells are useful in the therapy of any disease or disease in which adoptive transfer of T-cells are deemed beneficial, including, for example, treatment of cancer, treatment of pathogenic infection (e.g., viral disease such as HIV, bacterial infection, protozoan infection), treatment of inflammatory disorders (e.g., rheumatoid arthritis or Crohn's disease), and also for boosting the immune system.

In various embodiments, the methods disclosed herein can be used for the treatment of cancer. The term “cancer” is used herein to encompass any cancer, including but not limited to, melanoma, sarcoma, lymphoma, carcinoma such as brain, breast, liver, stomach and colon cancer, and leukaemia. In various embodiments, the methods disclosed herein can be used for treatment of a tumor. In various embodiments, the tumor is a solid tumor. In various embodiments the solid is a glioblastoma.

In various embodiments the tumor expresses a tumor associated antigen. Examples of such antigens include oncofetal antigens such as alphafetoprotein (AFP) and carcinoembryonic antigen (CEA), surface glycoproteins such as CA-125 and mesothelin, oncogenes such as Her2, melanoma-associated antigens such as dopachrome tautomerase (DCT), GP100 and MART1, cancer-testes antigens such as the MAGE proteins and NY-ESO1, viral oncogenes such as HPV E6 and E7, proteins ectopically expressed in tumours that are usually restricted to embryonic or extraembryonic tissues such as PLAC1, the ECM protein fibulin-3 which is expressed by GBM tumor cells but is absent in the brain and epidermal growth factor receptor (EGFR). As one of skill in the art will appreciate, an antigen may be selected based on the type of cancer to be treated using the present method as one or more antigens may be particularly suited for use in the treatment of certain cancers. For example, for the treatment of melanoma, a melanoma-associated antigen such as DCT may be used.

In various embodiments, the chimeric antigen receptor protein comprises interleukin 13 (IL13 CAR-T) or a variant thereof or a fragment thereof. In various embodiments, the nucleic acid encodes the interleukin 13 variant IL13.E13K.R109K or a fragment thereof. In various embodiments, the nucleic acid encodes a fragment of interleukin 13 comprising a domain that binds to an Interleukin 13 receptor or an extracellular domain thereof or a fusion protein comprising the Interleukin 13 receptor or the extracellular domain thereof. In various embodiments, the tumor antigen comprises an Interleukin 13 receptor (IL13R) or a variant thereof. In various embodiments, the tumor antigen comprises an alpha (α) chain of Interleukin 13 receptor (IL13Rα) or a variant thereof. In various embodiments, the chimeric antigen receptor protein comprises an extracellular domain capable of targeting fibulin 3.

In various embodiments disclosed herein, the chimeric antigen receptor (CAR) is directed toward a tumor associated antigen. In various embodiments the tumor associated antigen that the CAR is designed to target, is selected based on the type of tumor antigen expressed by the patient to be treated by the methods disclosed herein.

In a preferred embodiment, the disclosure relates to methods and compositions for manipulation of T-cells that have been primed by tumors (e.g., tumor infiltrating lymphocytes or TILs), which following manipulation, can be advantageously applied in killing tumor cells. Preferably, the manipulated T-cells are autologously transferred to the host to promote destruction of tumor cells.

In a particularly preferred embodiment, the disclosure relates to methods and compositions for generation of memory T-cells that are useful in carrying out one or more of the aforementioned therapeutic applications.

In a related embodiment, the disclosure relates to a method for ex vivo expansion of a T-cell, comprising, isolating a sample comprising T-cells from a subject; transducing the T-cells with a nucleic acid encoding a chimeric antigen receptor protein comprising a molecule that binds to a tumor antigen; and contacting the transduced T-cells with a GSK3β inhibitor and the tumor antigen to expand transduced T-cells. Preferably, the T-cells are transduced with a nucleic acid encoding a chimeric antigen receptor protein comprising interleukin 13 (IL13 CAR-T) or a variant thereof or a fragment thereof. Particularly, the nucleic acid encodes a CAR comprising the interleukin 13 variant IL13.E13K.R109K or a fragment thereof.

In a related embodiment, the disclosure relates to a method for ex vivo expansion of a T-cell, comprising, isolating a sample comprising T-cells from a subject; transducing the T-cells with a nucleic acid encoding a fragment of interleukin 13 comprising a domain that binds to an Interleukin 13 receptor or an extracellular domain thereof or a fusion protein comprising the Interleukin 13 receptor or the extracellular domain thereof; and contacting the transduced T-cells with a GSK3β inhibitor and the tumor antigen to expand transduced T-cells. Preferably, the tumor antigen comprises an Interleukin 13 receptor (IL13R) or a variant thereof. Especially, the tumor antigen comprises an alpha (α) chain of Interleukin 13 receptor (IL13Rα) or a variant thereof. The GSK3β inhibitor may be a small molecule inhibitor or a genetic inhibitor of GSK3β comprising siRNA, miRNA, antisense oligonucleotide, ddRNAi, or a dominant-negative inhibitor of GSK3 (GSK3DN). Preferably, the GSK3β inhibitor is a small molecule GSK3I3 inhibitor, e.g., SB216763, TWS-119, 1-Azakenpaullone or 6-bromoindirubin-3′-oxime (BIO). In various embodiments, the T-cells may be activated and expanded simultaneously or sequentially, e.g., activation followed by expansion or expansion followed by activation.

In various embodiments, the disclosure relates to a method for treating a tumor in a subject in need thereof, comprising administering, into the subject, an effective amount of a composition comprising a plurality of activated and/or expanded T-cells expressing a chimeric antigen receptor protein comprising a molecule that binds to a tumor antigen (CAR-T), wherein the activation comprises contacting the CAR-T cells with the tumor antigen and the expansion comprises contacting the activated CAR-T cells with a GSK3β inhibitor. For example, in some embodiments, the activated CAR-T cells preferably express a chimeric antigen receptor protein and the chimeric antigen receptor protein binds to a tumor antigen. In various embodiments, the T-cells are autologous T-cells. Particularly, the tumor antigen is interleukin 13 receptor (IL13R) or a ligand binding domain thereof and the chimeric antigen receptor protein comprises I113 or a variant thereof or a fragment thereof, e.g., which binds to the tumor antigen IL13R (α1 or α2). In various embodiments, the GSK3β inhibitor may be a small molecule inhibitor or a genetic inhibitor of GSK3β comprising siRNA, miRNA, antisense oligonucleotide, ddRNAi, or a dominant-negative inhibitor of GSK3 (GSK3DN). Preferably, the GSK3β inhibitor is a small molecule GSK3β inhibitor, e.g., SB216763, 1-Azakenpaullone, 6-bromoindirubin-3′-oxime (BIO) or TWS-119. Under various embodiments, the T-cells may be activated and expanded simultaneously or sequentially, e.g., activation followed by expansion or expansion followed by activation.

In various embodiments a method is provided for treating a tumor in a subject in need thereof, comprising administering, into the subject, an effective amount of a composition comprising a plurality of activated and/or expanded autologous T-cells expressing a chimeric antigen receptor protein (CAR-T cells) comprising an IL13 variant IL13.E13K.R109K, wherein the activation comprises contacting the CAR-T cells with the tumor antigen and the expansion comprises contacting the activated CAR-T cells with a small molecule GSK3β inhibitor, e.g., SB216763, 1-Azakenpaullone, 6-bromoindirubin-3′-oxime (BIO) or TWS-119, wherein the activated CAR-T cell expresses a chimeric antigen receptor protein and wherein the chimeric antigen receptor protein binds to a tumor antigen. Under various embodiments, the T-cells may be activated and expanded simultaneously or sequentially, e.g., activation followed by expansion or expansion followed by activation.

In a various embodiments, a method is provided for treating a glioma in a subject in need thereof, comprising administering, into the subject, an effective amount of a composition comprising a plurality of activated and/or expanded T-cells expressing a chimeric antigen receptor protein comprising a molecule that binds to a tumor antigen (CAR-T), wherein the activation comprises contacting the CAR-T cells with the tumor antigen and the expansion comprises contacting the activated CAR-T cells with a GSK3β inhibitor. Under this embodiment, the activated CAR-T cells preferably express a chimeric antigen receptor protein and the chimeric antigen receptor protein binds to a tumor antigen that is expressed in the glioma, e.g., IL13R or a variant thereof. In various embodiments, the glioma is glioblastoma multiforme (GBM), anaplastic astrocytoma or pediatric glioma. In some embodiments, the activation comprises contacting the CAR-T cells with the glioma tumor antigen and the expansion comprises contacting the activated CAR-T cells with a small molecule GSK3β inhibitor, wherein the activated CAR-T cell expresses the chimeric antigen receptor protein that binds to the glioma tumor antigen. The GSK3β inhibitor may be a small molecule inhibitor or a genetic inhibitor. In some embodiments, the GSK3β inhibitor is a small molecule e.g., SB216763, 1-Azakenpaullone, TWS-119, or 6-bromoindirubin-3′-oxime (BIO). Alternately or additionally, the GSK3β inhibitor is a genetic agent comprising siRNA, miRNA, antisense oligonucleotide, ddRNAi, or a dominant-negative inhibitor of GSK3 (GSK3DN).

In various embodiments, the disclosure relates to a method for generating tumor-specific memory T cells, comprising transducing T-cells isolated from a subject's biological sample with a nucleic acid encoding chimeric antigen receptor (CAR-T) comprising a molecule that binds to a tumor antigen; contacting the CAR-T cells with the tumor antigen and a GSK3β inhibitor; detecting a first marker specific to memory cells and a second marker specific for the tumor antigen, thereby generating tumor-specific memory T cells. Preferably, the CAR-T cells are transduced with a nucleic acid encoding IL13 or a fragment thereof or a variant thereof, e.g., IL13.E13K.R109K, wherein the CAR protein binds to the tumor antigen comprising IL13 receptor or a ligand-binding domain thereof. In various embodiments, the activation comprises contacting the CAR-T cells with the tumor antigen and the expansion comprises contacting the activated CAR-T cells with a small molecule GSK3β inhibitor, e.g., SB216763, 1-Azakenpaullone, TWS-119 or 6-bromoindirubin-3′-oxime (BIO). Under some embodiments, the marker specific for memory cells is selected from CD45RO+ and CD45RA+ and the marker specific for tumor antigen comprises expression, e.g., cell-surface expression, of a protein, which binds to the tumor antigen. In various embodiments, the tumor-specific CAR-T cells are specific for IL13R-positive tumor cells, as ascertained by a functional assay comprising binding to, and optionally destruction of, IL13R-positive cells. In various embodiments, the tumor-specific memory cells are CD8+ T-cells. In some embodiments, the CAR-T cells activated with the tumor antigen and expanded in the presence of the GSK3β inhibitor, which are further selected for memory T-cells, demonstrate increased specificity and memory towards tumor cells expressing the tumor antigen.

In various embodiments, a method is provided for generating tumor-specific memory T cells, comprising transducing T-cells isolated from a subject's biological sample with a nucleic acid encoding chimeric antigen receptor (CAR-T) comprising a molecule that binds to a tumor antigen; contacting the CAR-T cells with the tumor antigen and a GSK3β inhibitor; detecting a first marker specific to memory cells; a second marker specific for the tumor antigen; and a third marker for memory CAR-T cell homeostasis; thereby generating tumor-specific memory T cells. Under an embodiment, the third marker is IL13R expression, T-bet expression, and/or PD-1 expression in CAR T-cells, wherein increased T-bet expression and/or attenuated PD-1 expression indicates improved CAR-T cell homeostasis. Especially, the method provides improved T-cell homeostasis comprising reduced T cell exhaustion, sustained cytokine expression, T-cell clonal maintenance, and/or promotion of CAR-T memory development.

In various embodiments, the disclosure relates to a method of manipulating T-cells using the aforementioned transduction, activation, expansion and the optional selection steps, wherein the CAR-T cells activated with the tumor antigen and expanded in the presence of the GSK3β inhibitor, which are further selected for memory T-cells, demonstrate increased specificity and improved memory towards tumor cells expressing the tumor antigen and also exhibit improved CAR-T cell homeostasis. Especially, the method provides for an expanded population of activated CAR-T cells having improved T-cell homeostasis comprising reduced T cell exhaustion, sustained cytokine expression, T-cell clonal maintenance, and/or promotion of CAR-T memory development.

In various embodiments, a composition is provided comprising a T cell which expresses a chimeric antigen receptor protein (CAR-T cell) and a GSK3β inhibitor. Preferably, the T-cell expresses a chimeric antigen receptor protein comprising interleukin 13 (IL13 CAR-T) or a variant thereof or a fragment thereof. Especially, the T-cell expresses a chimeric antigen receptor protein comprising interleukin 13 variant IL13.E13K.R109K.

In various embodiments, a composition is provided comprising a T cell which expresses a chimeric antigen receptor protein (CAR-T cell), wherein the chimeric antigen receptor protein binds to a tumor antigen and a GSK3β inhibitor. Preferably, the T-cell expresses a chimeric antigen receptor protein comprising interleukin 13 (IL13 CAR-T) or a variant thereof or a fragment thereof. Especially, the T-cell expresses a chimeric antigen receptor protein comprising interleukin 13 variant IL13.E13K.R109K.

In various embodiments, a composition is provided comprising a T cell which expresses a chimeric antigen receptor protein (CAR-T cell) and a GSK3β inhibitor. In some embodiments, the compositions comprise a CAR-T cell and a small molecule GSK3β inhibitor, e.g., SB216763, 1-Azakenpaullone, TWS-119 or 6-bromoindirubin-3′-oxime (BIO). Alternately or additionally, the compositions comprise a CAR-T cell and a genetic agent comprising siRNA, miRNA, antisense oligonucleotide, ddRNAi, or a dominant-negative inhibitor of GSK3 (GSK3DN).

In various embodiments, a formulation is provided for separate administration comprising a T cell, which expresses a chimeric antigen receptor protein (CAR-T cell) and a GSK3β inhibitor. Preferably, the GSK3β inhibitor is a small molecule GSK3β inhibitor, e.g., SB216763, 1-Azakenpaullone, TWS-119, or 6-bromoindirubin-3′-oxime (BIO). Alternately or additionally, the formulations comprise a genetic agent comprising siRNA, miRNA, antisense oligonucleotide, ddRNAi, or a dominant-negative inhibitor of GSK3 (GSK3DN).

In various embodiments, the disclosure relates to a kit comprising, in one or more packages, a chimeric antigen receptor (CAR) encoding nucleic acid construct which encodes interleukin 13 (IL13 CAR-T) or a variant thereof or a fragment thereof; a GSK3β inhibitor; and optionally a first regent for transducing T-cells with said CAR nucleic acid construct; and further optionally, a second reagent for activating T-cells. Preferably, the kit includes the chimeric antigen receptor (CAR) encoding nucleic acid construct; the GSK3β inhibitor; the first regent for transducing T-cells with said CAR nucleic acid construct; and the second reagent for activating T-cells. Under this embodiment, the first agent is a retroviral vector. Still further under this embodiment, second reagent is IL13Rα2-Fc. Especially, the nucleic acid construct included in the kit encodes a chimeric antigen receptor protein comprising interleukin 13 variant IL13.E13K.R109K and GSK3β inhibitor included in the kit is SB216763, 1-Azakenpaullone, TWS-119, or 6-bromoindirubin-3′-oxime (BIO). Alternately or additionally, the kits comprise a genetic GSK3β inhibitor comprising siRNA, miRNA, antisense oligonucleotide, ddRNAi, or a dominant-negative inhibitor of GSK3 (GSK3DN).

### EXAMPLES

The structures, materials, compositions, and methods described herein are intended to be representative examples of the disclosure, and it will be understood that the scope of the disclosure is not limited by the scope of the examples. Those skilled in the art will recognize that the disclosure may be practiced with variations on the disclosed structures, materials, compositions and methods, and such variations are regarded as within the ambit of the disclosure.

### Example 1

### Choice of CAR-T

Modification of IL13 to IL13.E13K.R109K increases Affinity of IL13 Molecules towards IL13Rα2.

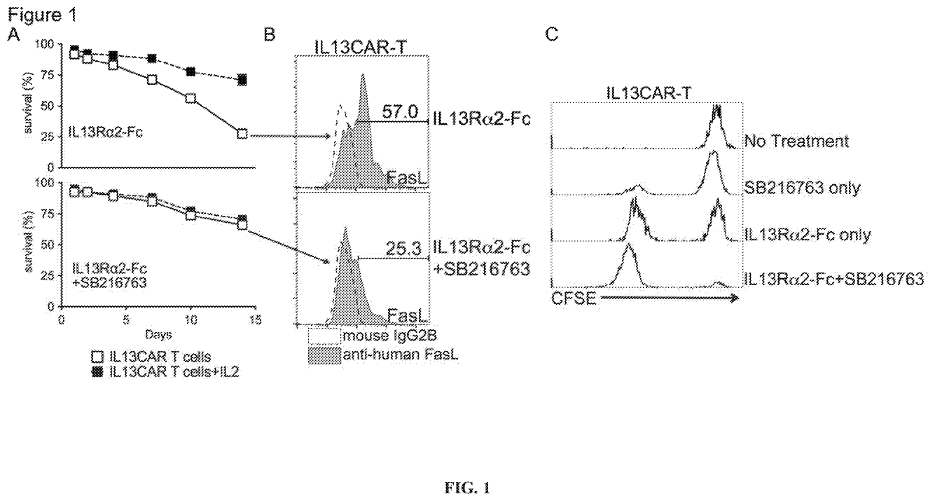

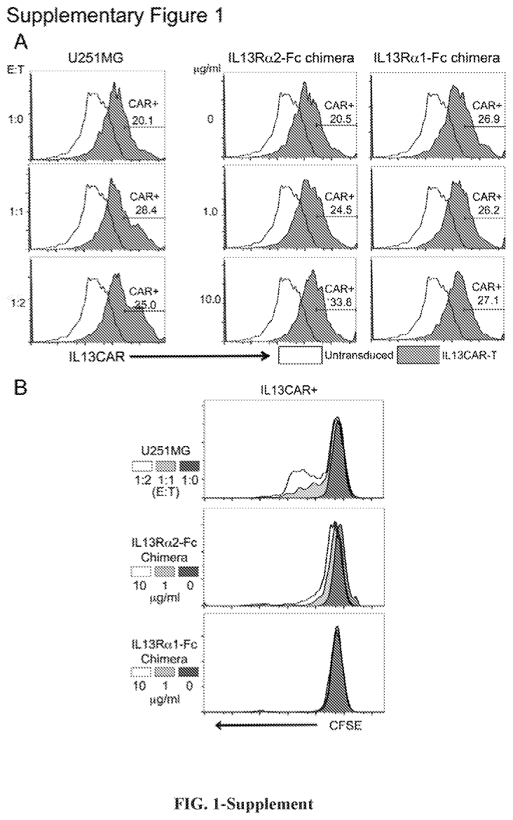

It has been shown that second generation CAR consisting of IL13.E13K.R109K as its extracellular ligand binding domain (IL13CAR-T) and intracellular CD28 costimulatory domain (IL13CAR-T) was successful in inducing specific cytotoxic response against IL13Rα2-expressing U251MG human glioma cell lines, and elimination of orthotopic tumors in xenograft glioma mouse model. Further, absolute specificity of IL13CAR-T to IL13Rα2 was shown in an experiment where IL13CAR-T when activated in presence of Mitomycin-C (50 μg/ml per 5×106 cells/ml for 20 minutes at 37° C.; Sigma, St. Louis, Mo.) treated U251MG glioma cells (at different ratios of T cells to Tumor cells) or with increasing concentrations IL13Rα2-Fc chimera (R&D Systems, Minneapolis, Minn.) showed CAR enrichment as well as IL13CAR-T proliferation. Similar observations were absent when IL13CAR-T cells were treated with increasing concentrations of IL13Rα1-Fc chimera—as high as 10 μg/m1 of the purified ligand (FIG. 1-Supplement). Therefore, IL13CAR-T was the chimeric antigen receptor (CAR) of choice for this study.

### IL13CAR Retrovirus Production and Modification of Primary Human T Cells

Preparation of retroviral supernatants containing IL13CAR expressing viral particles, and isolation of peripheral blood mononuclear cells (PBMCs) were performed as described previously (Beaudoin et al., J Virol Methods 148: 253-259, 2008). PBMCs were activated with OKT3 (100 ng/ml; Orthoclone) and IL2 (Proleukin, 3000 IU/ml; Prometheus Laboratories, San Diego, Calif.) for 48 hours.

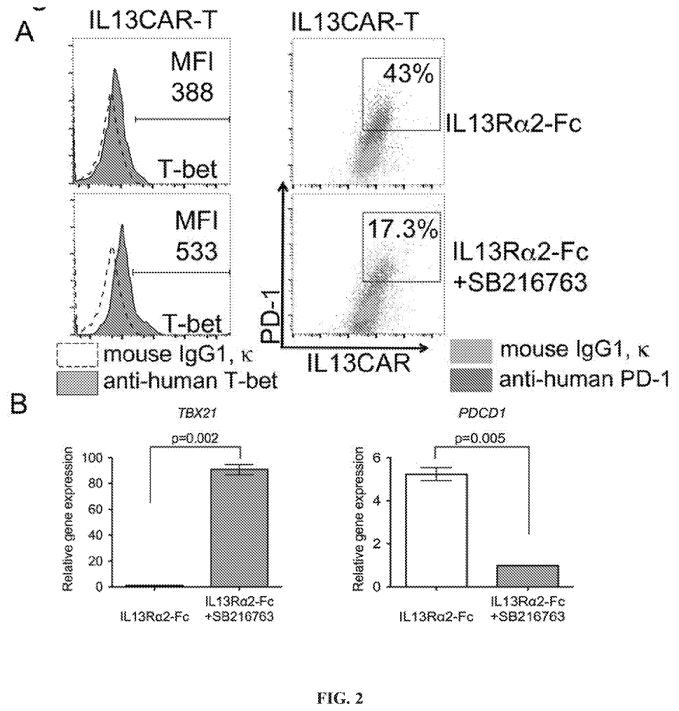

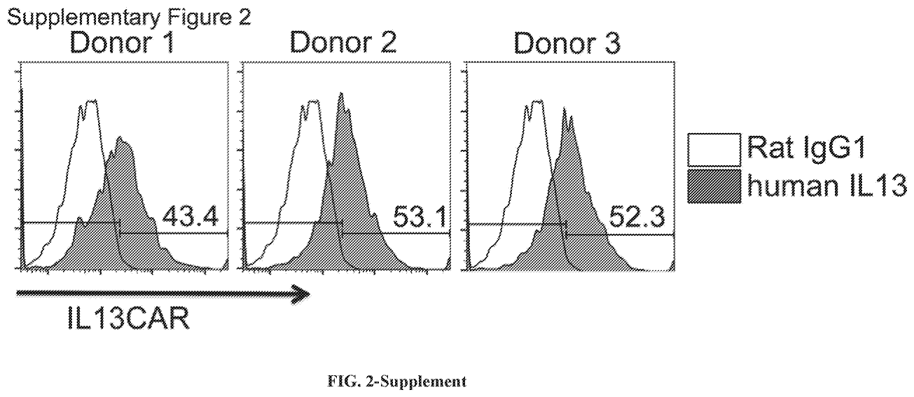

Enriched T-cells were transfected with retroviral supernatants using “spinfection” technique (Kong et al., Clin Cancer Res 18: 5949-5960, 2012). Transfected PBMCs were tested for IL13CAR expression (FIG. 2-supplement), and cultured in RPMI-1640 medium (Invitrogen, Grand Island, N.Y.) containing 10% FBS (Sigma, St. Louis Mo.), antibiotics and IL2, resulting 10-20-fold expansion and >95% Pure T cells. Activated untransduced T cells were used as control group in all experiments.

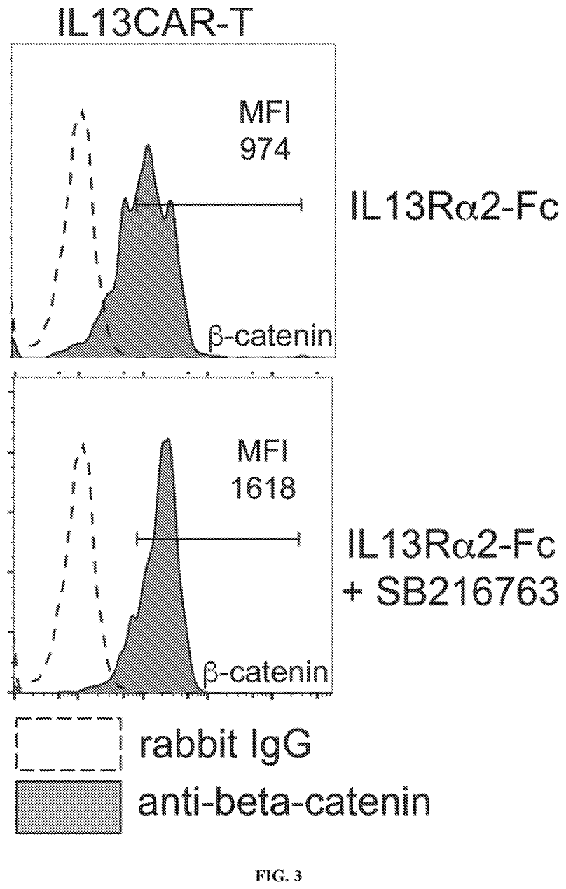

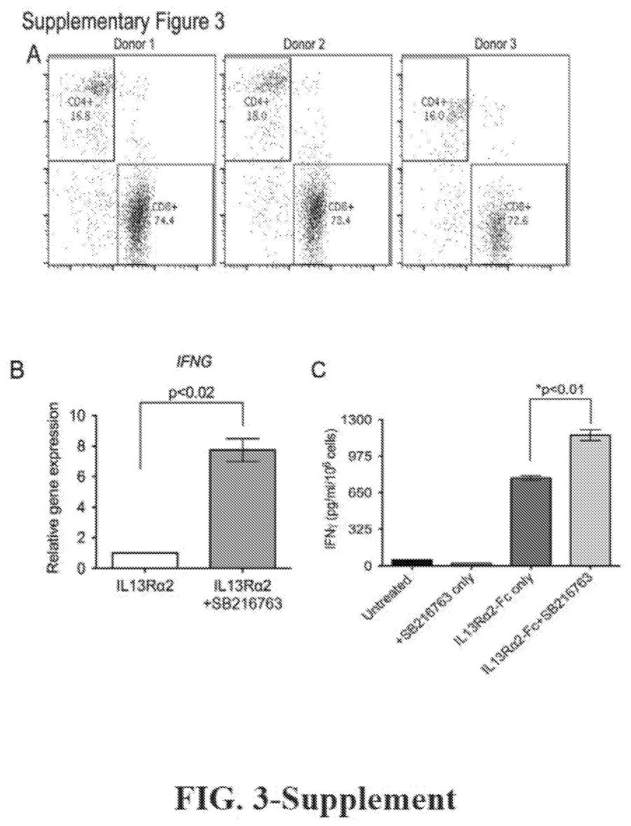

Experiments monitoring the types of T-cells generated during CAR-T cell expansion show CD8 enrichment. Activation of IL13CAR-T cells with IL13Rα2-Fc and GSK3β inhibition also showed a persistent CD8-enriched phenotype. Further, a 7.5-fold increase in IFNG gene expression and 2-fold increase in Interferon-gamma (IFNγ) secretion by activated IL13CAR-T cells that were treated with GSK3β inhibitors (FIG. 3-supplement) confirm CD8 enrichment in IL13CAR-T cells.

In order to mitigate donor variability, results of the aforementioned studies were controlled against IL13CAR expression.

Flow cytometric Analysis

Flow cytometry was performed using an LSRII instrument (BD Biosciences, San Jose, Calif.) and FACSDiva software (Version 6.2; BD Biosciences). All flow cytometric data were analyzed using FlowJo Software (Version 10.2; Flow Jo LLC, Ashland, Oreg.).

Purified rat anti-human IL13 antibody and allophycocyanin (APC)-conjugated anti-rat antibody was used to measure IL13CAR expression. Anti-human CD3-FITC was used in certain experiments for identifying T cells. For CD4:CD8 analysis of IL13CAR-T cells, anti-human CD4-FITC and anti-human CD8-PE.Cy7 were used in CAR-T cells that were positive for IL13CAR expression. FasL expression and PD-1 expression on activated IL13CAR-T cells was measured by staining with anti-human FasL-FITC (Thermo-Fisher) anti-human PD1-FITC respectively. Anti-human CD127-FITC, anti-human CD62L-PE, Anti-human CCR7-FITC anti-human CD45RO-PE and anti-human CD45RA-PE.Cy7 were used for flow cytometric measurement of T cell memory marker. Respective isotype controls or antibody controls (where applicable) were used to draw positive gates for each experiment. All antibodies were procured from either BD Biosciences or eBisociences. For intranuclear staining of β-catenin localization, nuclear permeabilization of CAR-T cells was achieved using FoxP3 staining buffer (eBioscience-Affymetrix, San Diego, Calif.) and staining with anti-human β-catenin rabbit mAb (Cell Signaling Technologies, Danvers Mass.) and anti-rabbit IgG conjugated with Alexa Fluor (AF) 488 or 647 (Cell Signaling Technologies). Cells that were not treated for nuclear permeabilization with FoxP3 staining buffer did not show any changes in β-catenin expression.

Carboxyfluorescein succinimidyl ester (CFSE; 0.5 μg/ml; Invitrogen) was used to measure T cell proliferation by flow cytometry.

### T Cell Survival Assays

Untransduced or IL13CAR-T cells (1×10⁶) were activated with IL13Rα2-Fc chimera at specific concentrations in 24 well plates, with or without GSK3β inhibitor (SB216763, 20 μM; Sigma, St Louis, Mo.) or added IL2 in the culture medium. IL13CAR-T cell survival assays were performed as described above for 14 days. Long-time survival of IL13CAR-T cells following GSK3β inhibition was measured by flow cytometry using live-dead gating (Sengupta et al., Immunobiology 210: 647-659, 2005). Activated T cell death (ATCD) was measured by flow cytometric reading of FasL expression (FITC; Thermo-Fisher) on activated IL13CAR-T cells.

Quatitative PCR (qPCR)

Total RNA was isolated from IL13CAR-T cells using the RNeasy Mini Kit according to the manufacturer's protocol (Qiagen). cDNA was prepared from RNA using iScript cDNA Synthesis Kit (Biorad, Carlsbad, Calif.). qPCR was performed targeting IFNG, TBX21, and PDCD1 genes using SyBR Green PCR master Mix (Applied Biosystems). C_{T} values of target genes were normalized to that of housekeeping gene GAPDH, and relative gene expression was calculated using ΔΔC_{T} method.

### ELISA

Culture supernatants were harvested from IL13CAR-T were activated with IL13Rα2-Fc±SB216763 for 72 h, and Interferon-gamma (IFNγ) levels were detected by ELISA using Ready-Set-Go ELISA detection kit (eBiocience, USA) according to manufacturer's protocol. OD values were measured using (Biotek, USA). Unactivated IL13CAR-T cells or those treated with SB216763 alone were used as experimental controls. Concentrations of IFNγ secreted by IL13CAR-T cells were extrapolated from standard curves drawn from respective experimental setup using measured OD values.

### In Vivo Immune Re-Challenge Study

Six-week old male athymic nude mice were purchased from JAX Mice (Bar Harbor, Me.). All mice were house in specific pathogen-free facility at the Roger Williams Medical Center, and experiments were conducted according to federal and institutional guidelines and with the approval of Roger Williams Medical Center Institutional Animal Care and Use Committee.

Forty-five animals were randomized, from which 40 animals were implanted with tumor cells, while 5 were chosen as experimental controls. Upper flanks of left hind limbs of each mouse were injected subcutaneously with 2×10⁶ IL13Rα2-expressing U251MG human glioma cells suspended in 200 μl phosphate buffered saline (PBS). Seven days after tumor implantation, tumor-bearing mice were randomized into 5 groups for treatment with 5×10⁶ IL13CAR-T cells in 50 μl of PBS (40% modification; n=10); or 5×10⁶ IL13CAR-T activated with IL13Rα2-Fc chimera (n=10); or 5×10⁶ IL13CAR-T activated with IL13Rα2-Fc chimera+SB216763 (n=10); or 5×10⁶ Untransduced T cells (n=5), or PBS only (n=5). Animals were observed for tumor growth, systemic and neurologic toxicity and death was recorded.

Sixty days after CAR-T treatment, the surviving animals were rechallenged with subcutaneous injections of 2×10⁶ U251MG glioma cells in 200 μl of PBS on the opposite flanks from the original tumor implantation. At day 100, the experiment was terminated and surviving animals were euthanized. Tumor tissue, draining lymph nodes (inguinal) and spleens were harvested from each animal for flow cytometric analysis of tumor-infiltrating IL13CAR-Ts and T cell memory markers.

The results of the experiments demonstrate the following:

GSK3β Inhibition Protects Activated CAR-T Cells from Activated T Cell Death (ATCD) in the Absence of IL2 Supplement

IL13CAR-T cells (32% CAR+) were cultured for 14 days in the presence of soluble IL13Rα2-Fc (1 μg/ml) and GSK3β inhibitor (SB216763; 20 μM) in RPMI1640 medium supplemented with 10% FBS and antibiotics, with or without added IL2. Cells were harvested at days 1, 4, 7, 10 and 14 and stained for CD3 and IL13CAR expression, and viability of cells were measured by flow cytometry and analyzed for viability as described earlier. In the absence of SB216763, IL13Rα2-Fc treated showed steady loss in viability indicating activated T cell death (FIG. 1A; Top Panel; open squares). The loss in viability was rescued by addition of IL2 in culture conditions (FIG. 1A; Top Panel; close squares) or inhibition of GSK3β with SB216763 in absence of added IL2 (FIG. 1A; Bottom Panel; open squares). Addition of IL2 in the presence of SB216763 in culture conditions did not have additive or synergistic effects on the viability of IL13CAR-T cells (FIG. 1A; Bottom Panel; closed squares). This indicated that inhibition of GSK3β in activated CAR-T cells promoted survival signaling, and suggested that GSK3β inhibition may protect activated CAR-T cells from ATCD in the absence of IL2 supplement. To confirm this phenomenon FasL expression was measured in IL13Rα2-Fc treated CAR-T cells at day 14. Observations concluded that SB216763 treatment reduced FasL expression by 55% in activated CAR-T cells (25.3%) in comparison to those that were not treated with the inhibitor (55%) confirming that indeed GSK3β inhibition protected activated CAR-T cells from ATCD (FIG. 1B). All other experiments in this study were performed in the absence of added IL2 in the culture conditions.

To further understand the mechanism, CAR-T cells were stained with CFSE and were cultured either unstimulated or treated with IL13Rα2-Fc±SB216763 for 72 hours. CFSE is a fluorescent cell staining dye and can be used to monitor lymphocyte proliferation, both in vitro and in vivo, due to the progressive halving of CFSE fluorescence within daughter cells following each cell division (Lyons et al., Journal of immunological methods 171: 131-137, 1994). GSK3β inhibition caused increased proliferation of IL13Rα2-Fc activated CAR-T cells only, while exerting no such efforts on unstimulated CAR-T cells (FIG. 1C). These results showed that GSK3β-inhibition resulted in increased expansion of IL13Rα2-activated IL13CAR-T cells, which was resultant of both functionalities of increased proliferation and enhanced survival of activated CAR-T cells.

### T-Bet Mediated Decrease in PD-1 Expression in Activated CAR-T Cells

GSK3 inhibition reduces PD-1 mediated T cells exhaustion, which is dependent on T-bet expression (Taylor et al., Immunity 44: 274-286. 2016), and GSK3β pathway directly regulates T-bet expression in activated T cells (Verma et al., J Immunol 197: 108-118, 2016). Significant survival advantage of GSK3β inhibition in activated T cells prompted us to study T-bet and PD-1 expression in IL13Rα2-activated IL13CAR-T cells. FACS analysis of activated IL13CAR-T cells showed significant upregulation of T-bet expression (FIG. 2A, left panel) while there were 60% reduction in PD-1 expression (17.3%) upon GSK3β inhibition, when compared to IL13CAR-T cells that were not treated with SB21673 (43%; FIG. 2A, right panel). qPCR analysis showed 90-fold increase in TBX21 gene (FIG. 2B, left panel) and 5-fold decrease in PDCD1 gene (FIG. 2B, right panel) upon GSK3β inhibition confirming that GSK3β inhibition induced T-bet mediated decrease PD-1 expression in activated CAR-T cells.

### GSK3β Inhibition Results in Increased Accumulation of β-Catenin in the Nucleus of Activated CAR-T Cells

Experiments were conducted to understand the molecular mechanism of GSK3β-inhibition on activated T cell expansion. GSK3β inhibition activates Wnt-signaling pathway by protecting β-catenin degradation (Lyons et al., Journal of immunological methods 171: 131-137, 1994). It has been previously shown in mouse models of T cell survival that GSK3β-inhibition increases activated T cell survival by increases in nuclear β-catenin expression (Sengupta et al., J Immunol 178: 6083-6091, 2007). IL13Rα2-Fc activated CAR-T cells were treated with or without SB216763 for 36-48 hours and measured for intra-nuclear accumulation of β-catenin by flow cytometry. GSK3β inhibition resulted in 66% increased accumulation of β-catenin (MFI 1618) in the nucleus of activated CAR-T cells over those that were not treated with SB216762. (MFI 974; FIG. 3).

### GSK3β Inhibition and Activated CAR-T Cell Memory Generation

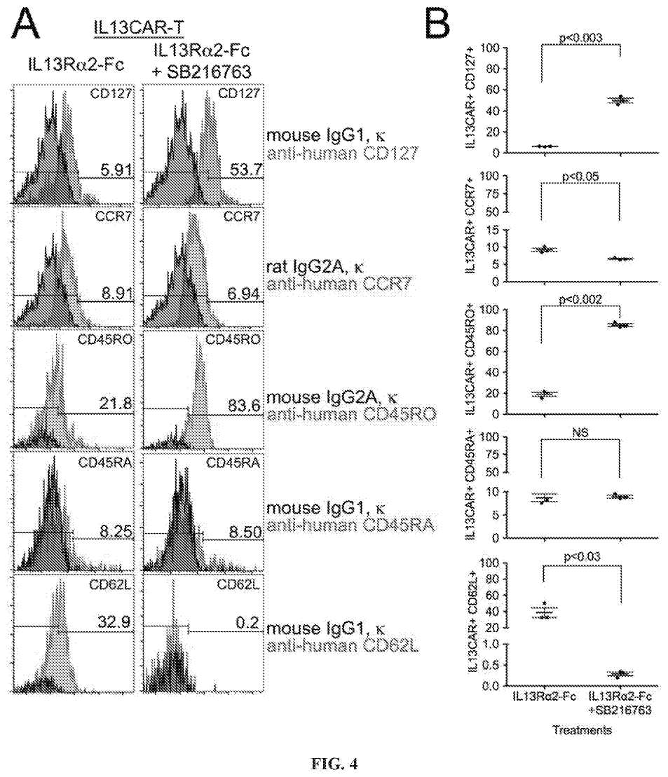

Recent studies have suggested a role played by intranuclear accumulation of β-catenin in development of CD8+ memory T cells (Gattinoni et al., Nat Med 15: 808-813, 2009; Taylor et al., Immunity 44: 274-286.2016; Verma et al., J Immunol 197: 108-118, 2016). Experiments were conducted to test the effects of SB216763 treatment on memory generation in IL13Rα2-Fc activated IL13CAR-T populations. IL13CAR-T cells were activated with increasing concentrations of IL13Rα2-Fc (0-1 μg/ml) in the presence or absence of SB216763 for 7 days. Cell surface expression of T cell memory markers were measured by flow cytometry. Since memory generation was monitored as a functional derivative of CD8+ T cells, experiments were conducted to additionally measure the intracellular expression of IL7R (CD127) expression as a marker of CD8+ memory CAR-T cell homeostasis. Analysis of flow cytometric data showed 10-fold increase in CD127 (FIG. 4A, FIG.4B, top panel) and 4-fold increase CD45RO (FIG. 4A, FIG.4B, third panel) in activated IL13CAR-T cells upon SB216763 treatment. This observation suggested that GSK3β inhibition induced intranuclear β catenin accumulation promoted a homeostatic proliferation of antigen-specific CD8+ effector T memory phenotype in activated CAR-T cells. However, there were no difference in CCR7 (FIG. 4A, FIG.4B, second panel) and CD45RA (FIG. 4A, FIG.4B, fourth panel), complete inhibition of CD62L expression (FIG. 4A, FIG.4B, bottom panel) suggested development of cell central memory phenotype in GSK3β inhibited antigen-specific CAR-T cells.

### Human Glioma Xenograft Tumor-Rechallenge Experiment and In Vivo Memory Development of SB216763-Treated Activated CAR-T Cells

Studies were conducted to test immune rechallenge effects of GSK3β inhibition in activated CAR-T cells in a xenograft glioma mouse model. Experiment was set up as described in Materials & Methods. Tumor growth was rapid in tumor-bearing animals treated with PBS [median survival (MS) 32 days] or untransduced T cells (MS 42 days), and animals had to be euthanized following approved IACUC protocol. Tumor regression was rapid and progression-free survival prolonged in groups of animals that were treated with CAR-T cells, irrespective of their activation status. This reflected a similar pattern as observed previously (Kong et al., Clin Cancer Res 18: 5949-5960, 2012). Those tumor-bearing animals that survived beyond 60 days post-implantation were rechallenged with a single injection of U251MG tumor cells on the opposite flanks from the original implantation. Tumor growth and animal survival was monitored, and the experiment was concluded on 100th day post-implantation following approved IACUC protocol. MS and overall survival of each experimental group were measured. At the conclusion of the experiment, tumor bearing animal groups that were treated with unactivated IL13CAR-T were 100% recurrent while those treated with IL13Rα2-Fc activated CAR-T were 67% recurrent. All surviving animals from the group that was treated with IL13Rα2-Fc+SB216763 activated IL13CAR-T were tumor-free (0% recurrent). Animal group that was treated with IL13CAR-T activated ex vivo with IL13Rα2-Fc+SB216763 had MS of 76.5 days. Four out of ten animals were alive in this group and all the surviving animals (0 of 4) were tumor-free.

### CAR-T Cell Memory Generation in Experimental Animals

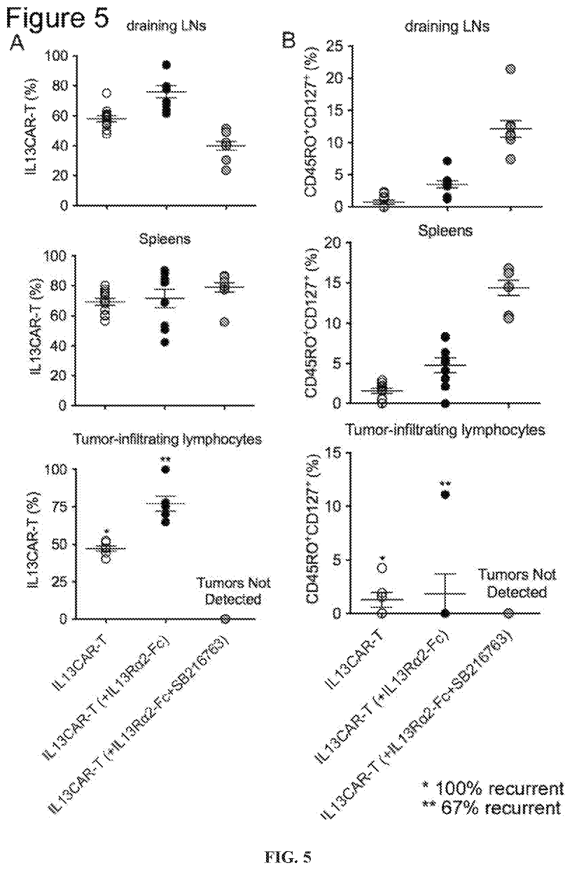

Tumors (where available), draining inguinal lymph nodes and spleen from each surviving animal from above were harvested. Single cell suspensions prepared from each organ were prepared and tested for tissue distribution of CAR-T cells and expression of immune memory markers. Cells were stained for human CD3 and IL13CAR (IL13CAR-T; FIG. 5A). Flow cytometric analysis showed 58% cells of draining lymph nodes (draining LN), 65% spleen cells and 48% Tumor-infiltrating lymphocytes (TIL) were IL13CAR-T+ in unactivated IL13CAR-T treated groups (open circles). In the group of animals that were treated with IL13CAR-T activated ex vivo with IL13Rα2-Fc only (closed circles), 75% of draining LN and TILs, and 65% of spleen cells were IL13CAR-T+. While only 30% of draining LNs and 70% of spleen cells stained positive for IL13CAR-T in animals that were treated with IL13CAR-T activated ex vivo with IL13Rα2-Fc+SB216763 (grey circles). TILs could not be studied because all the animals in this group were tumor-free. Flow cytometric analysis of CD45RO+CD127+ on IL13CAR-T cells (FIG. 5B) showed extremely low (<1 to 2%) frequency of antigen-specific CD8+ effector T memory in groups that were treated with unactivated IL13CAR-T and IL13CAR-T activated ex vivo with IL13Rα2-Fc only. Comparatively higher proportions of antigen-specific CD8+ effector T memory expression was observed on IL13CAR-T cells harvested from draining LNs (10%) and spleens (14%) of animals that were treated with IL13CAR-T activated ex vivo with IL13Rα2-Fc+SB216763. Incidentally, the treatment group with higher expressions of memory markers in peripheral lymphoid tissues was also the one where animals were tumor-free at the conclusion of the experiment.

Other embodiments: The preceding examples can be repeated with similar success by substituting the generically or specifically described reactants and/or operating conditions described elsewhere in the specification for those used in the preceding examples.

The exemplified embodiment makes use of lymphocytes, e.g., T-cells, comprising IL13CAR constructs (e.g., IL13.E13K.R109K). Detailed disclosure on the nucleic and/or amino acid sequences of such constructs including, methods for transducing T-cells with nucleic acids encoding the constructs is provided in Sengupta et al., U.S. Pat. No. 9,650,428 and Int. Pub. No. WO 2016/089916, the entirety of the disclosures therein, including, Drawings, Sequence Listings, and Tables showing relative mapping of the various constructs, are incorporated by reference herein.

The exemplified embodiment utilizes GSK3β inhibitors for improving the functionality of T-cells, specifically, CAR-T cells comprising a chimeric antigen receptor construct (e.g., IL13.E13K.R109K). The disclosure is not limited to the application of SB216763 (3-(2,4-Dichlorophenyl)-4-(1-methyl-1H-indol-3yl)-1H-pyrrole-2,5-dione)(Santa Cruz Biotech, Santa Cruz, Calif., USA) for this purpose. Other suitable GSK-3β inhibitors include, but are not limited to lithium, GF109203X (2-[1-(3-Dimethylaminopropyl)-1H-indol-3-yl]-3-(1H-indol-3-yl)maleimide), 1-Azakenpaullone (Sigma-Aldrich, Saint Louis, Mo., USA); 6-Bromoindirubin-3′-oxime (BIO)(Sigma-Aldrich, Saint Louis, Mo., USA); RO318220 (2-[1-(3-(Amidinothio)propyl)-1H-indol-3-yl]-3-(1-methylindol-3-yl)maleimide methanesulfonate); TWS-119 ((3-[6-(3-aminophenyl)-7H-pyrrolo[2,3-d]pyrimidin-4-yloxy]phenol; CAS#601514-19-6); Sigma Aldrich, St. Louis, Mo., USA); SB415286 (3-[(3-Chloro-4-hydroxyphenyl)amino]-4-(2-nitrophenyl)-1H-pyrrole-2,5-dione) (GlaxoSmithKline, London, United Kingdom); 4-Benzyl-2-methyl-1,2,4-thiadiazolidine-3,5-dione (“TDZD-8”) (Axxora, San Diego, Calif., USA); 2-Thio(3-iodobenzyl-5-(1-pyridyl)-[1,3,4]-oxadiazole (“TIBPO”) (Axxora, San Diego, Calif., USA); 2,4-Dibenzyl-5-oxothiadiazolidine-3-thione (“OTDZT”) (Axxora, San Diego, Calif., USA); and 4-(2-Amino-4-oxo-2-imidazolin-5-ylidene)-2-bromo-4,5,6,7-tetrahydropyrrolo [2,3-c]azepin-8-one (10Z-Hymenialdisine)(Axxora, San Diego, Calif., USA). In addition, a number of monoclonal antibodies directed to GSK-3β are commercially available from Axxora. Other pharmacological inhibitors of GSk-3β are set forth in Meijer et al., “Pharmacological Inhibitors of Glyocogen Synthase Kinase 3, Trends Pharmacol Sci. 2004 September; 25(9):471-80 (PUBMED #15559249), which is incorporated by reference in its entirety. See also U.S. Pat. Pub. No. 2007-0196514 to Li et al.

Although, in the exemplified embodiments, IL13CAR-T has been used as a candidate CAR because of previous successful preclinical studies (Kong et al., Clin Cancer Res 18: 5949-5960, 2012), the disclosure is not limited to the exemplified embodiments. The disclosed methods can be applied to any CAR-T therapy for solid tumors, where CAR-T cell access to tumor antigens is limited, resulting in weaker immune response. Representative examples of such tumors include, for example, glioblastoma multiforme (GBM), anaplastic astrocytoma and pediatric glioma.

In the embodiment exemplified above, activity of CAR-T against hyper-variable tumors such as glioblastoma multiforme (GBM) was investigated. GBM is an excellent model for studying antigen presentation by solid tumors. The Examples section of the instant disclosure examines activation, proliferation and successful memory generation of CAR-T cells. In hyper-variable tumors like GBM, unpredictability of antigenic profile plays an important role in success or failure of any immunotherapeutic regimen including CAR-T therapy, which can be addressed by targeting multiple tumor antigens. Alternately and/or additionally, a plurality of GBM neoantigens may be employed, including, antigens which are selected for personalized therapy, based on, for example, the level of expression in a particular patient or a patient class.

Although scientific literature generally points to weak exposure of CAR-T cells to antigens in solid tumors like GBM, use of GSK3β inhibitors conferred strong CAR-T cell proliferation, which was significant compared to controls and also surprising in the context of tumor therapy. The results showed increased proliferation of SB216763-treated activated CAR-Ts, which survived longer than those that were not activated in the presence of the GSK3β inhibitor. Addition of IL2 to the culture medium did not affect the viability of the GSK3β-inhibited CAR-T cells. Increased survival of activated IL13CAR-T cells that were treated with SB216763 was effected by lower expression of FasL in these T cells confirming that inhibition of GSK3β protected the activated CAR-T cells from activation-induced T cell death (ATCD). However, protection from ATCD was the not the only functional outcome of GSK3β inhibition on these T cells. Treatment with SB216763 resulted in increased proliferation of activated CAR-T cells, as observed in CFSE-profile of these cells. Yet, similar effect of GSK3β inhibition on T cell proliferation was not observed in unactivated CAR-T cells, which indicated that an adjuvant-like effect of GSK3β inhibition on activated or antigen-specific CAR-T cells.

Additionally, studies on exhaustion of activated CAR-T cells demonstrated 90-fold increase in T-bet gene (TBX21) and 5-fold reduction in PD-1 gene (PDCD1) expression with corresponding changes in protein expression upon SB216763 treatment of activated IL13CAR-T cells. These observations have strong significance in designing immunotherapies against solid tumors such as GBM. High levels of PD-1 on tumor-infiltrating T cells, including therapeutic CAR-T cells mark a subset of exhausted T cells with diminished effector function resulting from impaired proliferative, cytolytic, and cytokine production capabilities. PD-1 pathway blockade rescues these T cells from exhaustion, primarily with monoclonal antibodies targeting PD-1 or PD-L1 (expressed on target cells). Multiple clinical trials are ongoing where PD-1/PD-L1 targeting, as well as combinational immunotherapies with other immuno- and radiotherapy are being tested for treatment of GBM (Maxwell et al., Curr Treat Options Oncol 18: 51, 2017; Luksik et al., Neurotherapeutics, doi: 10.1007/s13311-017-0513-3, Mar. 3, 2017). Accordingly, embodiments of the instant disclosure provide for successful CAR-T cell immunotherapy of GBMs, comprising, for example, decreasing PD-1 expression on T cells by inhibiting GSK3β. Such a strategy may provide effective method of reducing T cell exhaustion, particularly of activated and/or proliferated CAR-T cells.

Embodiments described herein report a very distinct CD62L-negative CAR-T cell population that was also high expressers of CD45RO and T cell homeostatic marker IL7R or CD127 (CD62L⁻CD45RO⁺CD127³⁰ ). No changes were observed with respect to CD45RA expression upon GSK3β inhibition in activated CAR-T cells, which was consistent with the fact that CD45RA expression on human CD8⁺T cells is dependent on the original antigenic stimulation. These cells were low expressers of CCR7, which clearly indicated distinct CD8⁺ T effector memory (T_{EM}) development. In xenograft animal experiments, U251MG human glioma cell-bearing nude mice were treated with IL13CAR-T cells that were activated with—i) IL13Rα2-Fc in vitro, ii) with IL13Rα2-Fc+SB216763 in vitro, iii) with unactivated IL13CAR-T cells, or iv) untransduced T cells. Animals surviving beyond 60 days were rechallenged with tumor cells. Animals injected with IL13CAR-Tcells (with or without in vitro activation) cumulatively survived better than those that were either untreated or received untransduced T cells (median survival 42 days vs 76.5 days). However most importantly, all the surviving animals in the tumor bearing group that were treated with GSK3β-inhibited activated CAR-T cells (IL13Rα2-Fc+SB216763 in vitro) were tumor-free at the end of the experiment (100 days). Other surviving groups were either 100% recurrent (unactivated CAR-T) or 67% recurrent (2 of 3; IL13Rα2-Fc only in vitro). Analysis of CAR-T cell memory generation in vivo showed increased accumulation of CAR-T cells in draining lymph nodes and spleens of tumor bearing animals that were injected with unactivated or activated CAR-T that were not treated with GSK3β inhibitor (IL13Rα2-Fc only). Consistent with the expectations, these CAR-Ts were low expressers of CD45RO⁺IL7R⁺ phenotype. Interestingly, increased tumor-infiltrating IL13CAR-T cells were observed in groups that were treated with activated CAR-Ts (IL13Rα2-Fc only) than the group that received unactivated CAR-T cells.

This suggested increased tumor clearing efficiency of activated CAR-T cells, which was also reflected in the recurrence rate of 67% in comparison to 100% in unactivated CAR-T cell injected group. However, low levels of IL13CAR-T cells were observed in the lymph nodes of tumor-bearing animals that were treated with GS K3β-inhibited CAR-T cells (IL13Rα2-Fc+SB216763), while very highly present in spleens. No tumor-infiltrating lymphocytes were observed in these animals because they were all tumor-free. These CAR-T cells were higher expressers of CD45RO⁺IL7R⁺ phenotype, consistent with the fact that TEM cells are generally absent in lymph nodes and usually accumulate in spleens and other peripheral tissues. These in vivo results suggest vaccine-like effects of GSK3β inhibition on antigen-specific CAR-T cells.

The hallmark of successful immune response is when a) the immune system mounts an effective response to an antigen, and b) generates memory to recognize the same antigen in future. The exemplified embodiment shows for the first time that GSK3β inhibition promoting increased survival by mitigating ATCD and increasing proliferation in antigen-specific CAR-T cells and there by imparting the “immune-boost” required for successful immune response against solid tumors. The additional data demonstrating reduced CAR-T cell exhaustion by lowering PD-1 expression, and CD8⁺CAR-T_{EM} memory generation upon GSK3β inhibition in antigen-specific CAR-T cells, including subsequent clearance of tumors in experimental animals satisfies the second criteria. The adjuvant-like effects of GSK3β inhibition on antigen-experienced CAR-T cells provides for use of the compositions and methods of the disclosure (e.g., GSK3β inhibitor along with CAR-T) for the immunotherapy of cancers (more specifically, solid tumors) and also development of tumor vaccines.

Moreover, as the discovery of cancer neoantigens progresses, the embodiments disclosed herein can be modified for the development of new tumor vaccines based on CAR-T cells, which may be personalized in a disease-specific or patient specific-manner

From the foregoing description, one skilled in the art can easily ascertain the essential characteristics of the methods and, without departing from the spirit and scope thereof, can make various changes and modifications to adapt it to various usages and conditions.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described in the foregoing paragraphs. In addition, the materials, methods, and examples are illustrative only and not intended to be limiting. In case of conflict, the present specification, including definitions, will control.

All United States patents and published or unpublished United States patent applications cited herein are incorporated by reference. All published foreign patents and patent applications cited herein are hereby incorporated by reference. All published references, documents, manuscripts, scientific literature cited herein are hereby incorporated by reference. All identifier and accession numbers pertaining to scientific databases referenced herein (e.g., PUBMED, NCBI) are hereby incorporated by reference.

The following disclosures are incorporated by reference in their entireties:
1. Garfall et al., Chimeric Antigen Receptor T Cells against CD19 for Multiple Myeloma. N Engl J Med 373: 1040-1047, 2015.
2. Porter et al., Chimeric antigen receptor-modified T cells in chronic lymphoid leukemia. N Engl J Med 365: 725-733, 2011.
3. Yong et al., CART cell therapy of solid tumors. Immunol Cell Biol., 2016.
4. Newick et al., CART Cell Therapy for Solid Tumors. Annu Rev Med., 2016.
5. Yvon et al., Immunotherapy of metastatic melanoma using genetically engineered GD2-specific T cells. Clin Cancer Res 15: 5852-5860, 2009.
6. Emtage et al., Second-generation anti-carcinoembryonic antigen designer T cells resist activation-induced cell death, proliferate on tumor contact, secrete cytokines, and exhibit superior antitumor activity in vivo: a preclinical evaluation. Clin Cancer Res 14: 8112-8122, 2008.
7. Hombach et al., Costimulation by chimeric antigen receptors revisited the T cell antitumor response benefits from combined CD28-OX40 signalling. Int J Cancer 129: 2935-2944, 2011.
8. Brown et al., Regression of Glioblastoma after Chimeric Antigen Receptor T-Cell Therapy. N Engl J Med 375: 2561-2569, 2016.
9. van der Stegen et al., Preclinical in vivo modeling of cytokine release syndrome induced by ErbB-retargeted human T cells: identifying a window of therapeutic opportunity? J Immunol 191: 4589-4598, 2013.
10. Panelli et al., Forecasting the cytokine storm following systemic interleukin (IL)-2 administration. J Transl Med 2: 17, 2004.
11. Bonifant et al., Toxicity and management in CAR T-cell therapy. Mol Ther Oncolytics 3: 16011, 2016.
12. Gargett et al., GD2-specific CAR T Cells Undergo Potent Activation and Deletion Following Antigen Encounter but can be Protected From Activation-induced Cell Death by PD-1 Blockade. Mol Ther 24: 1135-1149, 2016.
13. Cherkassky et al., Human CAR T cells with cell-intrinsic PD-1 checkpoint blockade resist tumor-mediated inhibition. J Clin Invest 126: 3130-3144, 2016.
14. Pauken et al., Epigenetic stability of exhausted T cells limits durability of reinvigoration by PD-1 blockade. Science 354: 1160-1165, 2016.
15. Welsh et al., T-cell activation leads to rapid stimulation of translation initiation factor eIF2B and inactivation of glycogen synthase kinase-3. J Biol Chem 271: 11410-11413, 1996.
16. Ohteki et al., Negative regulation of T cell proliferation and interleukin 2 production by the serine threonine kinase GSK-3. J Exp Med 192: 99-104, 2000.
17. Sengupta et al., Unrestrained glycogen synthase kinase-3 beta activity leads to activated T cell death and can be inhibited by natural adjuvant. J Immunol 178: 6083-6091, 2007.
18. Gattinoni et al., Wnt signaling arrests effector T cell differentiation and generates CD8+ memory stem cells. Nat Med 15: 808-813, 2009.
19. Zhou et al., Differentiation and persistence of memory CD8(+) T cells depend on T cell factor 1. Immunity 33: 229-240, 2010.
20. Thaci et al., Significance of interleukin-13 receptor alpha 2-targeted glioblastoma therapy. Neuro Oncol 16: 1304-1312, 2014.
21. Kong et al., Suppression of human glioma xenografts with second-generation IL13R-specific chimeric antigen receptor-modified T cells. Clin Cancer Res 18: 5949-5960, 2012.
22. Beaudoin et al., Sorting vector producer cells for high transgene expression increases retroviral titer. J Virol Methods 148: 253-259, 2008.
23. Guha et al., Frontline Science: Functionally impaired geriatric CAR-T cells rescued by increased alpha5beta1 integrin expression. J Leukoc Biol 102: 201-208, 2017.
24. Sengupta et al., Adjuvant-induced survival signaling in clonally expanded T cells is associated with transient increases in pAkt levels and sustained uptake of glucose. Immunobiology 210: 647-659, 2005.
25. Lyons et al., Determination of lymphocyte division by flow cytometry. Journal of immunological methods 171: 131-137, 1994.
26. Taylor et al., Glycogen Synthase Kinase 3 Inactivation Drives T-bet-Mediated Downregulation of Co-receptor PD-1 to Enhance CD8(+) Cytolytic T Cell Responses. Immunity 44: 274-286. 2016.
27. Verma et al., LFA-1/ICAM-1 Ligation in Human T Cells Promotes Th1 Polarization through a GSK3beta Signaling-Dependent Notch Pathway. J Immunol 197: 108-118, 2016.
28. Ikeda et al., Axin, a negative regulator of the Wnt signaling pathway, forms a complex with GSK-3beta and beta-catenin and promotes GSK-3beta-dependent phosphorylation of beta-catenin. The EMBO journal 17: 1371-1384, 1998.
29. Jeannet et al., Essential role of the Wnt pathway effector Tcf-1 for the establishment of functional CD8 T cell memory. Proc Natl Acad Sci U S A 107: 9777-9782, 2010.
30. Forget et al., Stimulation of Wnt/ss-catenin pathway in human CD8+ T lymphocytes from blood and lung tumors leads to a shared young/memory phenotype. PLoS One 7: e41074, 2012.
31. Maxwell et al., Clinical Trials Investigating Immune Checkpoint Blockade in Glioblastoma. Curr Treat Options Oncol 18: 51, 2017.
32. Luksik et al., The Role of Immune Checkpoint Inhibition in the Treatment of Brain Tumors. Neurotherapeutics, 2017.
33. Gattinoni et al., A human memory T cell subset with stem cell-like properties. Nat Med 17: 1290-1297, 2011.
34. Staal et al., Wnt signaling is required for thymocyte development and activates Tcf-1 mediated transcription. Eur J Immunol 31: 285-293, 2001.
35. Ioannidis et al., The beta-catenin-TCF-1 pathway ensures CD4(+)CD8(+) thymocyte survival. Nat Immunol 2: 691-697, 2001.
36. Xu et al., Deletion of beta-catenin impairs T cell development. Nat Immunol 4: 1177-1182, 2003.
37. Zhao, D. M., S. Yu, X. Zhou, J. S. Haring, W. Held, V. P. Badovinac, J. T. Harty, and H. H. Xue. 2010. Constitutive activation of Wnt signaling favors generation of memory CD8 T cells. J Immunol 184: 1191-1199.
38. Hurton et al., Tethered IL-15 augments antitumor activity and promotes a stem-cell memory subset in tumor-specific T cells. Proc Natl Acad Sci U S A 113: E7788-E7797, 2016.
39. Carrasco et al., CD45RA on human CD8 T cells is sensitive to the time elapsed since the last antigenic stimulation. Blood 108: 2897-2905, 2006.
40. Huster et al., Selective expression of IL-7 receptor on memory T cells identifies early CD40L-dependent generation of distinct CD8+ memory T cell subsets. Proc Natl Acad Sci U S A 101: 5610-5615, 2004.
41. Huster et al., Unidirectional development of CD8+ central memory T cells into protective Listeria-specific effector memory T cells. Eur J Immunol 36: 1453-1464, 2006.
42. Kaech et al., Selective expression of the interleukin 7 receptor identifies effector CD8 T cells that give rise to long-lived memory cells. Nat Immunol 4: 1191-1198, 2003.
43. Boyman et al., Cytokines and T-cell homeostasis. Curr Opin Immunol 19: 320-326, 2007.
44. Sengupta et al., U.S. Pat. No. 9,650,428 entitled “METHODS AND COMPOSITIONS FOR TREATING CANCER.”
45. Sengupta et al., International Publication No. WO 2016/089916 of PCT/US2015/063267 entitled “METHODS AND COMPOSITIONS FOR TREATING CANCER.”

1. A method for ex vivo expansion of a population of T-cells, comprising contacting said T-cells with a GSK3β inhibitor.

2. The method of claim 1, wherein the T-cells are first transfected transduced with a chimeric antigen receptor protein comprising a molecule that binds to a tumor antigen prior to contacting said T-cells with a GSK3β inhibitor.

3. The method of claim 1, wherein the T-cells are isolated from a subject.

4. The method of claim 2, wherein the method further comprises contacting the transduced T-cells with a tumor antigen.

5. The method of claim 4, wherein the T-cells are contacted with a GSK3β inhibitor and the tumor antigen simultaneously.

6. The method of claim 2, wherein the T-cells are transduced with a nucleic acid encoding a chimeric antigen receptor protein comprising interleukin 13 (IL13 CAR-T) or a variant thereof or a fragment thereof.

7. The method of claim 6, wherein the nucleic acid encodes the interleukin 13 variant IL13.E13K.R109K or a fragment thereof.

8. The method of claim 6, wherein the nucleic acid encodes a fragment of interleukin 13 comprising a domain that binds to an Interleukin 13 receptor or an extracellular domain thereof or a fusion protein comprising the Interleukin 13 receptor or the extracellular domain thereof.

9. The method of claim 6, wherein the tumor antigen comprises an Interleukin 13 receptor (IL13R) or a variant thereof.

10. The method of claim 9, wherein the tumor antigen comprises an alpha (α) chain of Interleukin 13 receptor (IL13Rα) or a variant thereof.

11. The method of claim 1, wherein the GSK3β inhibitor is
(a) a chemical selected from SB216763, 1-Azakenpaullone, TWS-119 or 6-bromoindirubin-3′-oxime (BIO); and/or
(b) a genetic agent selected from micro RNA (miRNA), small interfering RNA (siRNA), DNA-directed RNA interfering (ddRNAi) oligonucleotide, an antisense oligonucleotide or a combination thereof.

12. The method of claim 1, wherein the T-cell is a helper T cell, a cytotoxic T cell, a memory T cell, a regulatory T cell, natural killer T cell, or a γδ T cell.

13. The method of claim 1, wherein the expanded T-cells are subsequently administered back into a patient in order to treat a disease.

14. The method of claim 13, wherein the disease is a cancer.

15. The method of claim 14, wherein the cancer is a solid tumor.

16. The method of claim 15, wherein the tumor expresses a tumor antigen.

17. The method of claim 1, wherein the method comprises:
a. isolating a sample comprising said T-cells from a subject;
b. transducing the population of T-cells with a nucleic acid encoding a chimeric antigen receptor protein comprising a molecule that binds to a tumor antigen; and
c. contacting the transduced T-cells with a GSK3β inhibitor.

18. A composition comprising a T cell which expresses a chimeric antigen receptor protein (CAR-T cell) and a GSK3β inhibitor.

19. The composition of claim 18, wherein the chimeric antigen receptor protein binds to a tumor antigen.

20. The composition of claim 18, wherein the T-cell expresses a chimeric antigen receptor protein comprising interleukin 13 (IL13 CAR-T) or a variant thereof or a fragment thereof.

21. The composition of claim 20, wherein the T-cell expresses a chimeric antigen receptor protein comprising interleukin 13 variant IL13.E13K.R109K.

22. The composition of claim 18, wherein the GSK3β inhibitor is a small molecule or a genetic agent.

23. The composition of claim 22, wherein the GSK3β inhibitor is a small molecule which is SB216763, 1-Azakenpaullone, TWS-119 or 6-bromoindirubin-3′-oxime (BIO); or a genetic agent which is siRNA, miRNA, antisense oligonucleotide, ddRNAi, or a dominant-negative inhibitor of GSK3 (GSK3DN).

24. The composition of claim 23, wherein the GSK3β inhibitor is a genetic agent selected from micro RNA (miRNA), small interfering RNA (siRNA), DNA-directed RNA interfering (ddRNAi) oligonucleotide, an antisense oligonucleotide or a combination thereof, and dominant-negative allele of GSK3 (GSK3DN).

25. (canceled)

26. (canceled)

27. (canceled)

28. (canceled)

29. (canceled)

30. (canceled)

31. (canceled)

32. A T-cell that has inhibited GSKβ expression or activity compared to a native or a wild-type T-cell.

33. The T-cell of claim 32, which is a helper T cell, a cytotoxic T cell, a memory T cell, a regulatory T cell, natural killer T cell, or a γδ T cell.

34. The T-cell of claim 32, wherein the T-cell comprises a genetic inhibitor comprising micro RNA (miRNA), small interfering RNA (siRNA), DNA-directed RNA interfering (ddRNAi) oligonucleotide, an antisense oligonucleotide or a combination thereof, wherein the genetic inhibitor inhibits activity or expression of GSK3β in the T-cell.

35. (canceled)

36. A method for ex vivo expansion of a T-cell, comprising, isolating a sample comprising T-cells from a subject; contacting the T-cells with a GSK3β inhibitor; transducing the T-cells with a nucleic acid encoding a chimeric antigen receptor protein comprising a molecule that binds to a tumor antigen; and contacting the transduced T-cells with the tumor antigen to activate and/or expand transduced T-cells.

37. The method of claim 36, wherein the T-cells are transduced with a nucleic acid encoding a chimeric antigen receptor protein comprising interleukin 13 (IL13 CAR-T) or a variant thereof or a fragment thereof.

38. The method of claim 37, wherein the nucleic acid encodes the interleukin 13 variant IL13.E13K.R109K or a fragment thereof.

39. The method of claim 37, wherein the nucleic acid encodes a fragment of interleukin 13 comprising a domain that binds to an Interleukin 13 receptor or an extracellular domain thereof or a fusion protein comprising the Interleukin 13 receptor or the extracellular domain thereof.

40. The method of claim 38, wherein the tumor antigen comprises an Interleukin 13 receptor (IL13R) or a variant thereof.

41. The method of claim 40, wherein the tumor antigen comprises an alpha (α) chain of Interleukin 13 receptor (IL13Rα) or a variant thereof.

42. The method of claim 36, wherein the GSK3β inhibitor is
(a) a chemical selected from SB216763, 1-Azakenpaullone, TWS -119 or 6-bromoindirubin-3′-oxime (BIO); and/or
(b) a genetic agent selected from micro RNA (miRNA), small interfering RNA (siRNA), DNA-directed RNA interfering (ddRNAi) oligonucleotide, an antisense oligonucleotide or a combination thereof.

43. The method of claim 36, wherein the T-cell is a helper T cell, a cytotoxic T cell, a memory T cell, a regulatory T cell, natural killer T cell, or a γδ T cell.

44. A method for treating a disease that is treatable by adoptive transfer of T-cells in a subject in need thereof, comprising administering, into the subject, an effective amount of a composition comprising a plurality of activated and expanded T-cells wherein the activation comprises contacting the CAR-T with an antigen and the expansion comprises contacting the activated CAR-T cells with a GSK3β inhibitor.

45. The method of claim 44, wherein the GSK3β inhibitor is
(a) a chemical selected from SB216763, TWS-119, 1-Azakenpaullone or 6-bromoindirubin-3′-oxime (BIO); and/or
(b) a genetic agent selected from micro RNA (miRNA), small interfering RNA (siRNA), DNA-directed RNA interfering (ddRNAi) oligonucleotide, an antisense oligonucleotide or a combination thereof.

46. The method of claim 44, wherein the disease is a tumor disease, a pathogenic disease selected from a bacterial disease, a viral disease and a protozoan disease, or an autoimmune disease.

47. A composition comprising a T cell which expresses a chimeric antigen receptor protein (CAR-T cell) and a GSK3β inhibitor.

48. A method for treating a tumor in a subject in need thereof, comprising administering, into the subject, an effective amount of a composition comprising a plurality of activated and expanded T-cells expressing a chimeric antigen receptor protein comprising a molecule that binds to a tumor antigen (CAR-T), wherein the activation comprises contacting the CAR-T with the tumor antigen and the expansion comprises contacting the activated CAR-T cells with a GSK3β inhibitor, wherein the activated CAR-T cell expresses a chimeric antigen receptor protein and wherein the chimeric antigen receptor protein binds to a tumor antigen.

49. The method of claim 48, wherein the T-cells are autologous T-cells.

50. The method of claim 48, wherein the tumor antigen is interleukin 13 receptor (IL13R) or a ligand binding domain thereof.

51. The method of claim 48, wherein the chimeric antigen receptor protein comprises Il13 or a variant thereof or a fragment thereof.

52. The method of claim 48, wherein the chimeric antigen receptor protein comprises the IL13 variant IL13.E13K.R109K.

53. The method of claim 48, wherein the GSK3β inhibitor is
(a) a chemical selected from SB216763, 1-Azakenpaullone, TWS-119, or 6-bromoindirubin-3′-oxime (BIO); and/or
(b) a genetic agent selected from micro RNA (miRNA), small interfering RNA (siRNA), DNA-directed RNA interfering (ddRNAi) oligonucleotide, an antisense oligonucleotide or a combination thereof.

54. The method of claim 48, wherein the T-cells are activated and expanded simultaneously or sequentially.

55. The method of claim 48, wherein the tumor is IL13R positive.

56. The method of claim 48, wherein the tumor is an IL13R positive glioma.

57. A method for generating tumor-specific memory T cells, comprising transducing T-cells isolated from a subject's biological sample with a nucleic acid encoding chimeric antigen receptor (CAR-T) comprising a molecule that binds to a tumor antigen; contacting the CAR-T cells with the tumor antigen and a GSK3β inhibitor; detecting a first marker specific to memory cells and a second marker specific for the tumor antigen, thereby generating tumor-specific memory T cells.

58. The method of claim 57, wherein the CAR-T cells are transduced with a nucleic acid encoding IL13 or a fragment thereof or a variant thereof.

59. The method of claim 58, wherein the CAR-T cells are transduced with a nucleic acid encoding the IL13 variant IL13.E13K.R109K.

60. The method of claim 59, wherein the tumor antigen is IL13 receptor or a ligand-binding domain thereof.

61. The method of claim 57, wherein the GSK3β inhibitor is
(a) a chemical selected from SB216763, 1-Azakenpaullone, TWS-119 or 6-bromoindirubin-3′-oxime (BIO); and/or
(b) a genetic agent selected from micro RNA (miRNA), small interfering RNA (siRNA), DNA-directed RNA interfering (ddRNAi) oligonucleotide, an antisense oligonucleotide or a dominant negative GSK3 inhibitor (GSK3DN) or a combination thereof.

62. The method of claim 57, wherein the marker specific for memory cells is selected from CD45RO+ and CD45RA+ and the marker specific for tumor antigen comprises expression of a protein which binds to the tumor antigen.

63. The method of claim 57, wherein the CAR-T cells are specific for IL13R-positive tumor cells, as ascertained by a functional assay comprising binding to, and optionally destruction of, IL13R-positive cells.

64. The method of claim 57, wherein the memory T-cells are CD8+ T-cells.

65. The method of claim 57, further detecting a third marker for memory CAR-T cell homeostasis.

66. The method of claim 57, wherein the third marker is IL13R expression, T-bet expression, and/or PD-1 expression.

67. The method of claim 66, wherein increased T-bet expression and/or attenuated PD-1 expression indicates improved CAR-T cell homeostasis.

68. The method of claim 67, wherein T-cell homeostasis comprises reduced T cell exhaustion, sustained cytokine expression, T-cell clonal maintenance, and/or promotion of CAR-T memory development.

69. The method of claim 57, wherein the CAR-T cells generated via activation with the tumor antigen and expansion in the presence of the GSK3β inhibitor demonstrate increased specificity and memory towards tumor cells expressing the tumor antigen.
